(19) United States
(12) Patent Application Publication
(10) Pub. No.: US 2021/0230650 A1
(43) Pub. Date: Jul. 29, 2021

(54) METHOD FOR THE PRODUCTION OF TRIACYLGLYCERIDES AND FATTY ACIDS

(71) Applicants: COMMISSARIAT A L'ENERGIE ATOMIQUE ET AUX ENERGIES ALTERNATIVES, Paris (FR); FERMENTALG, Libourne (FR); CENTRE NATIONAL DE LA RECHERCHE SCIENTIFIQUE, Paris (FR)

(72) Inventors: Alberto Amato, Grenoble (FR); Younes Dellero, Grenoble (FR); Juliette Jouhet, Seyssinet-Pariset (FR); Eric Marechal, Grenoble (FR); Fabrice Rebeille, Voreppe (FR)

(21) Appl. No.: 16/769,104

(22) PCT Filed: Jan. 30, 2019
(86) PCT No.: PCT/EP2019/052207

§ 371 Date: Jun. 2, 2020

(30) Foreign Application Priority Data
Jan. 31, 2018 (EP) 18305100.2

(51) Int. Cl. C12P 7/64; C12N 9/02

(52) U.S. Cl. CPC C12P 7/6427; C12P 7/6472; C12R 2001/89; C12Y 114/19005; C12N 9/0071

(57) ABSTRACT

The disclosure pertains to a method for the production of triacylglycerides (TAGs or Triacylglyerols) and fatty acids by the recombinant expression of a Δ11 fatty acid desaturase in protists.

The invention pertains to a method for the production of triacylglycerides (TAGs or Triacylglycerols) and fatty acids by the recombinant expression of a Δ11 fatty acid desaturase in protists.

Polyunsaturated fatty acids (PUFAs), such as the ω3-docosahexaenoic acid (DHA, 22:6) and the ω3-eicosapentaenoic acid (EPA, 20:5), have multiple benefits for human health (Simopoulos A P, Experimental Biology and Medicine, 233(6):674-88, 2008). In particular, DHA plays a crucial role in various biochemical processes and is necessary to the normal functional development of cells (for example, DHA is necessary for brain development in newborns and children). However, PUFAs are poorly synthesized in animals and are thus considered as essential fatty acids, which must be obtained by diet. Today, the most widely and naturally available diet source is fish oil but the overexploitation of fish stocks and the contamination by toxic substances (such as heavy metals) impose to find viable alternative sources. Since fishes obtain their ω3-fatty acids from zooplankton that consumes phytoplankton, microalgae and marine protists, like Thraustochytrids, appear to be a promising source of ω3-PUFAs (Ward O P & Singh A, Process Biochemistry, 40(12):3627-52, 2005; Adrame-Vega T C et al., Current Opinion in Biotechnology, 26:14-8, 2014).

The yields of TAGs and of PUFAs contained in these TAGs may considerably vary from one protist to another and also depend on the growth conditions. It is well known that nutrient-deprived growth media (especially nitrogen or phosphorus deprived media) trigger lipid accumulation in microalgae species such as Phaeodactylum tricornutum or Nannochloropsis gaditana (Jouhet J et al., PLoS One, 12(8):e0182423, 2017). However, nutrient deficiencies in these organisms are associated with a growth arrest and an accumulation of TAGs, often at the expense of membrane glycerolipids so that the total amount of lipids per gram of biomass does not increase.

To overcome this problem, many attempts are made on various algal models to engineer the biosynthesis of fatty acids in order to get high lipid levels in fast growing cell cultures with a high biomass. So far, best results were obtained by overexpressing a recombinant enzyme diacylglycerol acyltransferase, which is directly involved in TAG synthesis, leading to a doubling of the total fatty acid content with only a moderate decrease of the growth rate (Dinamarca J et al., Journal of Phycology, 53(2):405-14, 2017). However, the above and most widely-used algal models are very poor in DHA, which has a recognized nutritional importance and a strong potential in terms of therapeutic applications.

Today, only few attempts have been made to engineer Thraustochytrids (Aasen I M et al., Applied Microbiology and Biotechnology, 100(10):4309-21, 2016; Yan J F et al., Applied Microbiology and Biotechnology, 97(5):1933-9, 2013) with only limited effects on the TAGs and ω3-fatty acid production. For example, a Δ5 desaturase was overexpressed to increase EPA in Thraustochytrids, but addition in the external medium of the substrate of the enzyme (ETA, 20:4) is required to obtain some EPA production (Kobayashi T et al., Applied and Environmental Microbiology, 77(11):3870-6, 2011).

Therefore, there is a need for new tools to increase the production of TAGs and fatty acids with these microorganisms.

In this context, the Inventors have found that the expression of a recombinant Δ11 fatty acid desaturase from insect results in a higher rate of growth in protists, thus improving the biomass production, together with an increase of total fatty acids and TAGs, without affecting the fatty acid composition. Moreover, no exogenous lipid precursor is needed in the culture medium to trigger fatty acids and TAGs accumulation.

In an aspect, the invention thus relates to the use of a recombinant Δ11 fatty acid desaturase comprising or consisting of a sequence having at least 50% identity with the sequence SEQ ID NO: 1 for increasing the content of triacylglycerides and/or the content of fatty acids in a protist.

More specifically, the invention provides a method for producing triacylglycerides and/or fatty acids, wherein said method comprises a step of expression of a recombinant fatty acid Δ11 desaturase comprising or consisting of a sequence having at least 50% identity with the sequence SEQ ID NO: 1 in a protist.

The main benefits of the method of the invention are found in the fact that it induces not only an increase of the biomass of the cultivated protist but also an increase of the content of total TAGs and fatty acids per cell, without affecting the fatty acid composition and with no need of exogenous lipid precursor.

As used herein, the term “triacylglyceride” (TAG or Triacylglycerol) refers to a lipid consisting of three fatty acids esterified to glycerol. In a triacylglyceride, the glycerol may be linked to saturated and/or unsaturated fatty acids. The triacylglycerides produced in the invention preferably contain one, two or three unsaturated fatty acids. More preferred are triacylglycerides containing one, two or three polyunsaturated fatty acids.

Preferably, the fatty acids which are produced in the invention are polyunsaturated fatty acids.

As used herein, the term “polyunsaturated fatty acid” (PUFA) refers to a fatty acid (i.e. a carboxylic acid with an aliphatic chain) that contains more than one double bond in its backbone. PUFAs are derived from fatty acids with 4 to 22 carbon atoms. Preferably, the PUFAs produced in the invention are long chain polyunsaturated fatty acids (LCPUFA) which are derived from fatty acids with 16 to 22 carbon atoms. More preferably, the PUFAs produced in the invention are very long chain polyunsaturated fatty acids (VLCPUFA) which are derived from fatty acids with 20 to 22 carbon atoms.

The PUFAs produced in the invention may be bound in membrane lipids and/or in TAGs, but they may also occur as free fatty acids or else bound in the form of other fatty acid esters. In this context, they may be present as pure products or in the form of mixtures of various fatty acids or mixtures of different glycerides.

In the invention, the PUFAs as free fatty acids or bound in the TAGs have preferably a chain length of at least 16 carbon atoms, more preferred are LCPUFA and VLCPUFA, even more preferred are eicosapentaenoic acid (EPA, 20:5), docosapentaenoic acid (DPA, 22:5), or docosahexaenoic acid (DHA, 22:6).

As used herein, “Eicosapentaenoic acid” (EPA) designates a PUFA which contains 20 carbons and 5 double bonds (20:5).

As used herein, “Docosapentaenoic acid” (DPA) designates a PUFA which contains 22 carbons and 5 double bonds (22:5).

As used herein, “Docosahexaenoic acid” (DHA) designates a PUFA which contains 22 carbons and 6 double bonds (22:6).

As used herein, the term “Δ11 fatty acid desaturase” (or detail fatty acid desaturase) refers to an enzyme which is capable of introducing a double bond at the 11^{th} position from the carboxyl end into fatty acids or their derivatives, such as fatty acyl-CoA esters. In particular, the Δ11 fatty acid desaturase used in the invention is a Δ11 acyl-CoA desaturase, which means it is capable of using acyl-CoA fatty acids as substrate. More preferred is a Δ11 acyl-CoA desaturase that is capable of desaturating acyl-CoA molecules with a chain length of 16 carbons or more.

In the method of the invention, the amino acid sequence of the recombinant Δ11 fatty acid desaturase expressed in a protist is an exogenous enzyme which may originate from insects, in particular from moths.

Indeed, the desaturases expressed in pheromone glands of different moth species play a key role in the biosynthesis of sex pheromones, exhibiting a wide variety of substrate and region- and stereo-specificities. In particular, pheromone gland desaturases catalyze the formation of uncommon unsaturated fatty acyl-CoA esters with variable chain lengths and either the ordinary Z or the unusual E double bond geometry.

Preferably, the amino acid sequence of the Δ11 fatty acid desaturase used in the invention originates from the Lepidoptera family, in particular selected from the group consisting of Acrolepiidae, Agaristidae, Arctiidae, Bombycidae, Carposinidae, Cochylidae, Cossidae, Eriocraniidae, Gelechiidae, Geometridae, Gracillariidae, Hepialidae, Ithomiidae, Lasiocampidae, Lycaenidae, Lymantriidae, Lyonetiidae, Nepticulidae, Noctuidae, Notodontidae, Nymphalidae, Oecophoridae, Papilionidae, Pieridae, Psychidae, Pterophoridae, Pyralidae, Saturniidae, Sesiidae, Sphingidae, Tortricidae, Yponomeutidae and Zygaenidae. More preferably, the amino acid sequence of the Δ11 fatty acid desaturase used in the invention originates from the genera Thaumetopoea, Helicoverpa or Spodoptera, and in particular selected from the species Thaumetopoea pityocampa, Helicoverpa zea or Spodoptera littoralis.

In an embodiment, the recombinant Δ11 fatty acid desaturase comprises or consists of a sequence having at least 50%, 51%, 52%, 53%, 54%, 55%, 56%, 57%, 58%, 59%, 60%, 61%, 62%, 63%, 64%, 65%, 66%, 67%, 68%, 69%, 70% 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with the sequence SEQ ID NO: 1.

As used herein, the “percentage identity” (or “% identity”) between two sequences of nucleic acids or amino acids means the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of the local homology algorithm of Smith and Waterman, by means of the similarity search method of Pearson and Lipman (1988) or by means of computer software using these algorithms (GAP, BESTFIT, FASTA and TFASTA in the Wisconsin Genetics Software Package, Genetics Computer Group, 575 Science Dr., Madison, Wis., or by the comparison software BLAST NR or BLAST P). The percentage identity between two nucleic acid or amino acid sequences is determined by comparing the two optimally-aligned sequences in which the nucleic acid or amino acid sequence to compare can have insertions or deletions compared to the reference sequence for optimal alignment between the two sequences. Percentage identity is calculated by determining the number of positions at which the amino acid, nucleotide or residue is identical between the two sequences, preferably between the two complete sequences, dividing the number of identical positions by the total number of positions in the alignment window and multiplying the result by 100 to obtain the percentage identity between the two sequences.

In an embodiment, the recombinant fatty acid Δ11 desaturase comprises or consists of a sequence selected from the group comprising SEQ ID NO: 1, SEQ ID NO: 2 and SEQ ID NO: 3, preferably SEQ ID NO: 1.

TABLE 1
Amino acid sequences of three reference Δ11 Acyl-CoA desaturases from
Thaumetopoea pityocampa, Helicoyerpa zea or Spodoptera littoralis
respectively.

| | |
|---|---|
| SEQ ID NO: 1 | MAPNTRENETIYDEVEHKLEKLVPPQAGPWNYKIVYLNLLTFSYWLI |
| Δ11 Acyl-CoA desaturase | AGAYGLYLCFTSAKWATIIFEFILFFFAEMGITAGAHRLWTHKSYKA |
| from Thaumetopoea | KLPLEIFLMVLNSVAFQNTATDWVRDHRLHHKYSDTDADPHNAARGL |
| pityocampa (UniProt | FFSHVGWLLVRKHDEVKKRGKFTDMSDIYNNPVLKFQKKYAIPFIGA |
| A8QVZ1) | VCFILPTVIPMYFWGESLNNAWHICILRYAMNLNVTFSVNSLAHIWG |
| | NKPYDKDIKPAQNFGVTLATFGEGFHNYHHVFPWDYRTSELGDNKFN |
| | FTTKFINFFERIGLAYDLKTVSDDVIAQRAKRTGDGTHLWDCADKNN |
| | NDVVQTKAQIDTLCTKHE |
| SEQ ID NO: 2 | MAQSYQSTTVLSEEKELTLQHLVPQASPRKYQIVYPNLITFGYWHIA |
| Δ11 Acyl-CoA desaturase | GLYGLYLCFTSAKWATILFSYILFVLAEIGITAGAHRLWAHKTYKAK |
| from Helicoyerpa zea | LPLEILLMVFNSIAFQNSAIDWVRDHRLHHKYSDTDADPHNASRGFF |
| (UniProt Q9NB26) | YSHVGWLLVRKHPEVKKRGKELNMSDIYNNPVLRFQKKYAIPFIGAV |
| | CFALPTMIPVYFWGETWSNAWHITMLRYIMNLNVTFLVNSAAHIWGN |
| | KPYDAKILPAQNVAVSVATGGEGFHNYHHVFPWDYRAAELGNNSLNL |
| | TTKFIDLFAAIGWAYDLKTVSEDMIKQRIKRTGDGTDLWGHEQNCDE |
| | VWDVKDKSS |
| SEQ ID NO: 3 | MAQCVQTTTILEQKEEKTVTLLVPQAGKRKFEIVYFNIITFAYWHIA |
| Δ11 Acyl-CoA desaturase | GLYGLYLCFTSTKWATVLFSFFLFVVAEVGVTAGSHRLWSHKTYKAK |
| from Spodoptera littoralis | LPLQILLMVMNSLAFQNTVIDWVRDHRLHHKYSDTDADPHNASRGFF |
| (UniProt Q6US81) | YSHVGWLLVRKHPDVKKRGKEIDISDIYNNPVLRFQKKYAIPFIGAV |
| | CFVLPTLIPVYGWGETWTNAWHVAMLRYIMNLNVTFLVNSAAHIYGK |
| | RPYDKKILPSQNIAVSIATFGEGFHNYHHVFPWDYRAAELGNNSLNF |
| | PTKFIDFFAWIGWAYDLKTVSKEMIKQRSKRTGDGTNLWGLEDVDTP |
| | EDLKNTKGE |

In an embodiment, the sequence of the recombinant Δ11 fatty acid desaturase used in the invention contains three highly conserved His-rich boxes consisting of SEQ ID NO: 4 (HRLW[T/A/S]H), SEQ ID NO: 5 ([D/E]HR[L/M/F/S]HH[K/R]) and SEQ ID NO: 6 ([F/S]HNYHH[V/T]) respectively.

In a particular embodiment, the recombinant Δ11 fatty acid desaturase can be in the form of a fragment (or a truncated sequence) of a sequence having at least 50% identity with SEQ ID NO: 1. Such a fragment of the Δ11 fatty acid desaturase preferably exhibits the same, or substantially the same, activity compared to the full length Δ11 fatty acid desaturase.

The desaturase activity can be verified by cultivating the microorganism expressing the recombinant enzyme, digesting it in a suitable buffer or solvent, bringing the digest into contact with fatty acids or acyl-CoA fatty acids and, if appropriate, with a cofactor such as NADH or NADPH or oxygen, and detecting the resulting desaturated fatty acids or acyl-CoA fatty acids. The fatty acids or acyl-CoA fatty acids can preferably originate from the transformed organism if it is itself capable of synthesizing fatty acids or acyl-CoA fatty acids. If not, however, it is also possible to add fatty acids or acyl-CoA fatty acids. The fatty acid or acyl-CoA fatty acid which has been modified by the desaturase or conjugase can be detected via customary methods with which the skilled work is familiar, if appropriate after extraction from the incubation mixture, for example with a solvent such as ethyl acetate. Separation methods such as high-performance liquid chromatography (HPLC), gas chromatography (GC), thin-layer chromatography (TLC) and detection methods such as mass spectroscopy (MS or MALDI), UV spectroscopy or autoradiography may be employed for this purpose.

In the invention, the expression of the recombinant Δ11 fatty acid desaturase takes place in protists.

As used herein, the term “protists” refers to the one-celled eukaryotic microorganisms classified in the taxonomic kingdom Protista. Protists are not animals, plants, fungi, yeast or bacteria. In the invention, suitable protists are autotroph or heterotroph, preferably heterotroph.

In an embodiment, the expression of the recombinant Δ11 fatty acid desaturase of the invention takes place in a protist which is a microalgae.

As used herein, the expression “microalgae” refers to microscopic algae, with sizes from a few micrometers to a few hundred micrometers.

In particular, the expression “microalgae” covers the microalgae with high industrial potential (for example used as food supplements or used for biofuel production): such as Nannochioropsis gatidana, Phaeodactylum tricornutum and Thalassiosira pseudonana.

In an embodiment, the expression of the recombinant Δ11 fatty acid desaturase of the invention takes place in a protist which is selected from the phylogenetic group SAR.

In an embodiment, the expression of the recombinant Δ11 fatty acid desaturase of the invention takes place in a protist which is selected from the supergroup Chromalveolata (Adl S M et al., Journal of Eukaryotic Microbiology, 52(5):399-451, 2005).

In the invention, the supergroup “Chromalveolata” refers to organisms within the clade kingdom of Chromista (Cryptista, Heterokonta, Haptophyta) and Alveolata. Amongst Heterokonta, important clade includes the Thraustochytrids (e.g. Auranthiochytrium), the Diatoms (e.g. Phaeodactylum) and the Eustigmatophytes (e.g. Nannochioropsis).

In an embodiment, the expression of the recombinant Δ11 fatty acid desaturase of the invention takes place in a protist which is a traustochytrid (Thraustochytriidae family), preferably from a genus selected from the group consisting of Aurantiochytrium, Japonochytrium, Sicyoidochytrium, Ulkenia, Parietichytrium, Botryochytrium, Schizochytrium, Monorhizochytrium and Thraustochytrium.

In particular, Aurantiochytrium is a thraustochytrid genus defined by Yokohama and Honda in 2007 (Yokoyama R. & Honda D, Mycoscience, 48:199-211, 2007, which is incorporated herein by reference for the purpose of defining the genus Aurantiochytrium, in particular last paragraph of page 207).

The genus Aurantiochytrium is characterized by the absence of well-developed ectoplasmic nets and a lower number of zoospores produced by each zoosporangium compared to the genus Schizochytrium. Molecular analyses of the 18S rDNA region and chemotaxonomical observations has revealed a clear separation of the two taxa. Moreover, Schizochytrium only synthesizes β-carotene as main pigment and between 15 and 30% of arachidonic acid (AA 20:4 ω6), whereas Aurantiochytrium can produce besides β-carotene, astaxantin, cantaxanthin and its intermediates and the main fatty acid is DHA with very low levels of AA.

Preferably, the expression of the recombinant Δ11 fatty acid desaturase of the invention takes place in a protist selected from the species Aurantiochytrium limacinum and Aurantiochytrium mangrovei.

The invention is preferably carried out in Auranthiochytrium limacinum (formerly Schizochytrium limacinum), a heterotrophic marine protist naturally rich in DHA (>30-40% of total fatty acids), which emerged as a micro algal model.

In a particular embodiment, the invention relates to a method for producing triacylglycerides and/or fatty acids, wherein said method comprises a step of expression of a recombinant fatty acid Δ11 desaturase comprising or consisting of a sequence having at least 50%, 51%, 52%, 53%, 54%, 55%, 56%, 57%, 58%, 59%, 60%, 61%, 62%, 63%, 64%, 65%, 66%, 67%, 68%, 69%, 70% 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with the sequence SEQ ID NO: 1, in a Thraustochytrid, preferably from a genus selected from the group consisting of Aurantiochytrium, Japonochytrium, Sicyoidochytrium, Ulkenia, Parietichytrium, Botryochytrium, Schizochytrium, Monorhizochytrium and Thraustochytrium, more preferably from the species Aurantiochytrium limacinum.

In an embodiment, the invention relates to the method as defined above, wherein the fatty acids are polyunsaturated fatty acids, which have preferably a chain length of 16 carbons or more.

In an embodiment, the invention relates to the method as defined above, wherein the polyunsaturated fatty acids have a chain length of 16, 18, 20 or 22 carbons.

In an embodiment, the invention relates to the method as defined above, wherein the polyunsaturated fatty acids are ω3-polyunsaturated fatty acids.

As used herein, “ω3-polyunsaturated fatty acids” (ω3-PUFAs or omega3-polyunsaturated fatty acids) are PUFAs with a double bond at the third carbon atom from the methyl end of the carbon chain.

In an embodiment, the invention relates to the method as defined above, wherein the ω3-PUFAs have a chain length of 16, 18, 20 or 22 carbons.

Preferred ω3-PUFAs are EPA, DPA and DHA.

In an embodiment, the invention relates to the method as defined above, wherein the TAGs contain at least one ω3-PUFA, said at least one ω3-PUFA having preferably 16 carbons or more.

In an embodiment, the invention relates to the method as defined above, wherein the TAGs contain at least one ω3-PUFA, said at least one ω3-PUFA being preferably selected from EPA, DPA and DHA.

In the invention, the content of total TAGs and fatty acids in the protist cell expressing the recombinant Δ11 fatty acid desaturase is increased compared to the content of total TAGs and fatty acids in the wild type protist cell grown in the same conditions.

In particular, the quantity of TAGs per cell (or per liter of culture or per liter of culture per day) of the protist expressing the recombinant Δ11 fatty acid desaturase is increased by at least a factor 1.1 compared to the quantity of TAGs per cell (or per liter of culture or per liter of culture per day) of the wildtype protist. Preferably the production of TAGs in the protist expressing the recombinant Δ11 fatty acid desaturase is increased by a factor 1.5, 2.0, 2.5, 3.0 or higher.

In particular, the quantity of fatty acids per cell (or per liter of culture or per liter of culture per day) of the protist expressing the recombinant Δ11 fatty acid desaturase is increased by at least a factor 1.1 compared to the quantity of fatty acids per cell (or per liter of culture or per liter of culture per day) of the wildtype protist. Preferably the production of fatty acids in the protist expressing the recombinant Δ11 fatty acid desaturase is increased by a factor 1.5, 2.0, 2.5, 3.0 or higher.

In an embodiment, the content of PUFAs in the protist cell expressing the recombinant Δ11 fatty acid desaturase is increased compared to the content of PUFAs in the wild type protist cell grown in the same conditions.

In particular, the quantity of PUFAs per cell (or per liter of culture or per liter of culture per day) of the protist expressing the recombinant Δ11 fatty acid desaturase is increased by at least a factor 1.1 compared to the quantity of PUFAs per cell (or per liter of culture or per liter of culture per day) of the wildtype protist. Preferably the production of PUFAs in the protist expressing the recombinant Δ11 fatty acid desaturase is increased by a factor 1.5, 2.0, 2.5, 3.0 or higher.

In particular, the quantity of DHA per cell (or per liter of culture or per liter of culture per day) of the protist expressing the recombinant Δ11 fatty acid desaturase is increased by at least a factor 1.1 compared to the quantity of DHA per cell (or per liter of culture or per liter of culture per day) of the wildtype protist. Preferably the production of DHA in the protist expressing the recombinant Δ11 fatty acid desaturase is increased by a factor 1.5, 2.0, 2.5, 3.0 or higher.

In particular, the quantity of DPA per cell (or per liter of culture or per liter of culture per day) of the protist expressing the recombinant Δ11 fatty acid desaturase is increased by at least a factor 1.1 compared to the quantity of DPA per cell (or per liter of culture or per liter of culture per day) of the wildtype protist. Preferably the production of DPA in the protist expressing the recombinant Δ11 fatty acid desaturase is increased by a factor 1.5, 2.0, 2.5, 3.0 or higher.

In particular, the quantity of EPA per cell (or per liter of culture or per liter of culture per day) of the protist expressing the recombinant Δ11 fatty acid desaturase is increased by at least a factor 1.1 compared to the quantity of EPA per cell (or per liter of culture or per liter of culture per day) of the wildtype protist. Preferably the production of EPA in the protist expressing the recombinant Δ11 fatty acid desaturase is increased by a factor 1.5, 2.0, 2.5, 3.0 or higher.

Another benefit of the present invention is that the expression of the recombinant Δ11 fatty acid desaturase in a protist induces not only an increase of the production of TAGs and fatty acids per cell but also an increase of the growth of the protist, and thus an increase of the biomass produced after culture.

In particular, a protist expressing the recombinant Δ11 fatty acid desaturase has a higher rate of growth compared to the wild type protist. For example, after 5 days of culture, the biomass of the protist expressing the recombinant Δ11 fatty acid desaturase is increased by at least a factor 1.1, preferably at least a factor 1.5, compared to the biomass of the wild type protist grown in the same conditions.

In an embodiment, the invention relates to a method as defined above, which further comprises a step of culture of the protist expressing the recombinant fatty acid Δ11 desaturase.

The culture of the protists is generally carried out in heterotrophic mode, preferably in chemically defined media. Some chemically defined culture media that can be used in the invention contain a carbon source, a nitrogen source and salts necessary to microorganism growth. The person skilled in the art knows well the elements necessary to microorganism growth.

For example, Traustochytrids, such as Auranthiochytrium limacinum, can be cultivated in a R medium as defined in Table 5 in the examples.

Auranthiochytrium limacinum is generally cultivated at a temperature between 20° C. and 30° C., preferably at 25° C.

Auranthiochytrium limacinum can also be cultivated at low temperatures, such as 15° C., since some studies have taught that low temperatures can increase its production of DHA.

In an embodiment, the invention relates to the method as defined above, wherein said method further comprises a step of lipid extraction from the culture of the protist expressing the recombinant fatty acid Δ11 desaturase.

After the protists have been cultured, the lipids are obtained in the customary manner. To this end, the protists can first be digested or else used directly. The lipids are advantageously extracted with suitable solvents such as apolar solvents, such as hexane or ethanol, isopropanol or mixtures such as hexane/isopropanol, phenol/chloroform/isoamyl alcohol, at temperatures between 0° C. to 80° C., preferably between 20° C. and 50° C. Some appropriate methods are presented in the examples.

In another aspect, the invention relates to oils, fatty acid mixtures and/or TAG mixtures, in particular with an increased content of PUFAs, which have been produced by the above-described method, and to their use for the production of foodstuffs, feedstuffs, cosmetics or pharmaceuticals. To this end, they are added in customary amounts to the foodstuffs, feedstuffs, cosmetics or pharmaceuticals.

In another aspect, the invention relates to a nucleic acid encoding a fatty acid Δ11 desaturase comprising or consisting of a sequence having at least 50% identity with the sequence SEQ ID NO: 1, said nucleic acid being codon-optimized for the expression of said fatty acid Δ11 desaturase in a protist.

In an embodiment, the invention relates to a nucleic acid encoding a fatty acid Δ11 desaturase comprising or consisting of a sequence having at having at least 50%, 51%, 52%, 53%, 54%, 55%, 56%, 57%, 58%, 59%, 60%, 61%, 62%, 63%, 64%, 65%, 66%, 67%, 68%, 69%, 70% 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% identity with the sequence SEQ ID NO: 1, said nucleic acid being codon-optimized for the expression of said fatty acid Δ11 desaturase in a protist.

In an embodiment, the invention relates to a nucleic acid encoding a fatty acid Δ11 desaturase comprising or consisting of a sequence having at least 50% identity with the sequence SEQ ID NO: 1, said nucleic acid being codon-optimized for the expression of said fatty acid Δ11 desaturase in a microalgae.

In an embodiment, the invention relates to a nucleic acid encoding a fatty acid Δ11 desaturase comprising or consisting of a sequence having at least 50% identity with the sequence SEQ ID NO: 1, said nucleic acid being codon-optimized for the expression of said fatty acid Δ11 desaturase in a protist which is selected from the phylogenetic group SAR, which comprises Stramenopiles, Alveolates and Rhizaria (Burki F et al., PLoS One. 2(8):e790, 2007).

In an embodiment, the invention relates to a nucleic acid encoding a fatty acid Δ11 desaturase comprising or consisting of a sequence having at least 50% identity with the sequence SEQ ID NO: 1, said nucleic acid being codon-optimized for the expression of said fatty acid Δ11 desaturase in a protist which is selected from the supergroup Chromalveolata.

In an embodiment, the invention relates to a nucleic acid encoding a fatty acid Δ11 desaturase comprising or consisting of a sequence having at least 50% identity with the sequence SEQ ID NO: 1, said nucleic acid being codon-optimized for the expression of said fatty acid Δ11 desaturase in a Traustochytrid, preferably from a genus selected from the group consisting of Aurantiochytrium, Japonochytrium, Sicyoidochytrium, Ulkenia, Parietichytrium, Botryochytrium, Schizochytrium, Monorhizochytrium and Thraustochytrium. more preferably from the species Aurantiochytrium limacinum and Aurantiochytrium mangrovei.

In an embodiment, the invention relates to a nucleic acid as defined above which comprises or consists of the sequence SEQ ID NO: 7.

TABLE 2
Example of a codon-optimized nucleic acid sequence
encoding the fatty acid Δ11 desaturase.

| | |
|---|---|
| SEQ ID NO: 7 | ATGGCGCCCAACACCCGCGAGAACGAGACCATCTACGATGAGGTTGA |
| | GCATAAGCTCGAGAAACTCGTGCCTCCTCAAGCGGGCCCCTGGAACT |
| | ACAAAATCGTTTATCTCAACCTTCTCACCTTCTCTTACTGGCTTATC |
| | GCCGGCGCCTACGGTCTCTATCTCTGTTTTACCTCCGCAAAATGGGC |
| | CACCATCATCTTCGAGTTCATCCTCTTCTTTTTCGCCGAGATGGGCA |
| | TCACCGCAGGTGCTCACCGCCTCTGGACCCATAAATCTTACAAAGCC |
| | AAGCTCCCTCTCGAGATCTTCCTCATGGTGCTCAATTCTGTTGCGTT |
| | CCAAAACACGGCCACGGACTGGGTGCGCGATCATCGCCTCCACCATA |
| | AGTACTCCGACACCGACGCGGATCCTCACAACGCTGCGCGCGGTCTC |
| | TTCTTCTCCCATGTCGGTTGGCTCCTCGTCCGCAAGCACGACGAGGT |
| | CAAGAAGCGCGGTAAGTTTACCGATATGTCCGATATCTACAACAATC |
| | CCGTGCTCAAGTTCCAGAAGAAATATGCCATCCCCTTCATCGGTGCC |
| | GTTTGCTTTATCTTACCTACCGTGATCCCCATGTACTTTTGGGGTGA |
| | GTCCCTCAACAACGCCTGGCACATCTGTATCCTCCGCTATGCGATGA |
| | ACCTCAACGTCACCTTCTCCGTGAACTCCCTCGCGCATATCTGGGGT |
| | AATAAGCCCTACGACAAGGATATCAAACCCGCTCAGAACTTCGGTGT |
| | TACCCTCGCGACCTTCGGTGAGGGTTTTCACAACTATCACCACGTGT |
| | TCCCCTGGGACTATCGCACCTCCGAGCTCGGCGACAACAAGTTCAAT |
| | TTCACCACCAAATTCATCAATTTCTTTGAGCGCATCGGTCTCGCGTA |
| | TGATCTCAAGACCGTTTCCGATGACGTTATCGCGCAACGCGCCAAAC |
| | GCACCGGTGATGGTACCCATCTCTGGGATTGCGCCGATAAGAATAAT |
| | AACGATGTTGTTCAAACCAAAGCGCAAATCGATACCCTCTGCACCAA |
| | ACATGAGTACCCCTACGACGTGCCCGACTACGCCTAACATATGCCAT |
| | GGTGTCAAAACCGGGGTTAGTGACATTGACTTGTTGACAAAAATCTG |
| | TATAGCTAGAAAACTCTAAGCAACGCTTTTCTTTGTTTTATTTTTTA |
| | TGTTTAAACTCCTTCAGAATTGTAGGATATCTTGTTTTGAAAAATCC |
| | AGGACTGAGTTTCGTTGCCCCATTTGCTTGTTCTCGTTTGAAATGTC |
| | GAACAATAGAAATGCTTGCAGAATGA |

The nucleic acid sequence SEQ ID NO: 7 has been codon-optimized to encode the amino acid sequence SEQ ID NO: 1 in Aurantiochytrium limacinum.

For example, a sequence encoding a fatty acid Δ11 desaturase from an insect can be codon optimized to be expressed in Aurantiochytrium using the codon usage table shown in Table 3.

TABLE 3
Codon usage table for heterologous expression in Aurantiochytrium
(based on 30192 residues of A. limacinum).

| Codon | Aminoacid | A. limacinum Percentage |
|---|---|---|
| TAA | * (stop) | 52.50% |
| TAG | * (stop | 20.20% |
| TGA | * (stop) | 27.40% |
| GCA | A | 31.40% |
| GCC | A | 21.20% |
| GCG | A | 14.40% |
| GCT | A | 33.00% |
| TGC | C | 55.40% |
| TGT | C | 44.60% |
| GAC | D | 43.20% |
| GAT | D | 56.80% |
| GAA | E | 47.70% |
| GAG | E | 52.30% |
| TTC | F | 41.10% |
| TTT | F | 58.90% |
| GGA | G | 25.70% |
| GGC | G | 30.30% |
| GGG | G | 11.70% |
| GGT | G | 32.40% |
| CAC | H | 47.70% |
| CAT | H | 52.30% |
| ATA | I | 11.20% |
| ATC | I | 35.60% |
| ATT | I | 53.20% |
| AAA | K | 42.30% |
| AAG | K | 57.70% |
| CTA | L | 8.60% |
| CTC | L | 21.70% |
| CTG | L | 13.10% |
| CTT | L | 30.40% |
| TTA | L | 9.30% |
| TTG | L | 16.90% |
| ATG | M | 100.00% |
| AAC | N | 51.70% |
| AAT | N | 48.30% |
| CCA | P | 28.80% |
| CCC | P | 18.90% |
| CCG | P | 17.10% |
| CCT | P | 35.30% |
| CAA | Q | 51.70% |
| CAG | Q | 48.30% |
| AGA | R | 11.90% |
| AGG | R | 8.50% |
| CGA | R | 16.20% |
| CGC | R | 27.50% |
| CGG | R | 9.40% |
| CGT | R | 26.60% |
| AGC | S | 17.50% |
| AGT | S | 15.00% |
| TCA | S | 18.50% |
| TCC | S | 13.70% |
| TCG | S | 11.80% |
| TCT | S | 23.60% |
| ACA | T | 30.40% |
| ACC | T | 23.80% |
| ACG | T | 15.90% |
| ACT | T | 29.90% |
| GTA | V | 18.50% |
| GTC | V | 22.30% |
| GTG | V | 26.50% |
| GTT | V | 32.60% |
| TGG | W | 100.00% |
| TAC | Y | 52.20% |
| TAT | Y | 47.80% |

Using an appropriate codon usage table, the expression of an exogenous enzyme in a given microorganism (such as a protist) can be boosted by replacing the original codons by the codons which are the most frequently used by said protist.

In another aspect, the invention relates to an expression cassette comprising a nucleic acid encoding a fatty acid Δ11 desaturase as defined above under the control of a promoter which is functional in a protist.

In an embodiment, the invention relates to an expression cassette comprising a nucleic acid encoding a fatty acid Δ11 desaturase as defined above under the control of a promoter which is functional in a microalgae.

In an embodiment, the invention relates to an expression cassette comprising a nucleic acid encoding a fatty acid Δ11 desaturase as defined above under the control of a promoter which is functional in a protist which is selected from the phylogenetic group SAR.

In an embodiment, the invention relates to an expression cassette comprising a nucleic acid encoding a fatty acid Δ11 desaturase as defined above under the control of a promoter which is functional in a protist which is selected from the supergroup Chromalveolata.

In an embodiment, the invention relates to an expression cassette comprising a nucleic acid encoding a fatty acid Δ11 desaturase as defined above under the control of a promoter which is functional in a Traustochytrid, preferably from a genus selected from the group consisting of Aurantiochytrium, Japonochytrium, Sicyoidochytrium, Ulkenia, Parietichytrium, Botryochytrium, Schizochytrium, Monorhizochytrium and Thraustochytrium. more preferably from the species Aurantiochytrium limacinum and Aurantiochytrium mangrovei.

In an embodiment, the invention relates to an expression cassette as defined above which comprises or consists of the sequence SEQ ID NO: 8.

TABLE 4
Example of a recombinant cassette for the expression of the
fatty acid Δ11 desaturase in Aurantiochytrium limacinum.

| | |
|---|---|
| SEQ ID NO: 8 | CTGCAGGTAGGTAGGTGGCAGTAGCGTTACGAGGAGGAGTCCCGAGA |
| | GGGAGTCGGAGAGTAGAAAACTGGAAGTCGGCGAAACAAAAGGCGCA |
| | GAGATTTGCCGGAATGGAGAGTTATCGTGAGACTCTCTGAGTAGACC |
| | CAAGTGTCCTGTGAGGCACTCGTGATAGGGAGGGGGCACGGGCTGAA |
| | GGGGGCTACAGTAAGGAGAGAGTGGCGTCAGTGGGGTTTTGCCGAGA |
| | ACTCTTCGAGAAAGAGGAAGAGAGGAACCGAGAGCGCCGTTGAAGAG |
| | GGGAAAAAGCAGACGGTTTAATTATAATTAATTAAGTAATTAATTAC |
| | TTACTTATTGATTGATTGATTTGAGAAGAGAAGCAAAGAGAGAGTTG |
| | AAGAAATAGTAACGAAGAATAGGAGAAGAAAGGGGCAAGAAAAGAAA |
| | AAGAAAGAGGAGAATATTAGTCGATGAGCGAGAACGTGCAAATCCAA |
| | AACAGCAAAACTCAAACTCAAACTCAAACTACAAGAAGCGTGGCGTT |
| | GCAGAGGCAACAGCTCGAAAGCAACACAGAACAAACAAACACAGGAG |
| | AGGCAGTAAGGTCAATTTCGCGGCCGCGCTAGCATGGCGCCCAACAC |
| | CCGCGAGAACGAGACCATCTACGATGAGGTTGAGCATAAGCTCGAGA |
| | AACTCGTGCCTCCTCAAGCGGGCCCCTGGAACTACAAAATCGTTTAT |
| | CTCAACCTTCTCACCTTCTCTTACTGGCTTATCGCCGGCGCCTACGG |
| | TCTCTATCTCTGTTTTACCTCCGCAAAATGGGCCACCATCATCTTCG |
| | AGTTCATCCTCTTCTTTTTCGCCGAGATGGGCATCACCGCAGGTGCT |
| | CACCGCCTCTGGACCCATAAATCTTACAAAGCCAAGCTCCCTCTCGA |
| | GATCTTCCTCATGGTGCTCAATTCTGTTGCGTTCCAAAACACGGCCA |
| | CGGACTGGGTGCGCGATCATCGCCTCCACCATAAGTACTCCGACACC |
| | GACGCGGATCCTCACAACGCTGCGCGCGGTCTCTTCTTCTCCCATGT |
| | CGGTTGGCTCCTCGTCCGCAAGCACGACGAGGTCAAGAAGCGCGGTA |
| | AGTTTACCGATATGTCCGATATCTACAACAATCCCGTGCTCAAGTTC |
| | CAGAAGAAATATGCCATCCCCTTCATCGGTGCCGTTTGCTTTATCTT |
| | ACCTACCGTGATCCCCATGTACTTTTGGGGTGAGTCCCTCAACAACG |
| | CCTGGCACATCTGTATCCTCCGCTATGCGATGAACCTCAACGTCACC |
| | TTCTCCGTGAACTCCCTCGCGCATATCTGGGGTAATAAGCCCTACGA |
| | CAAGGATATCAAACCCGCTCAGAACTTCGGTGTTACCCTCGCGACCT |
| | TCGGTGAGGGTTTTCACAACTATCACCACGTGTTCCCCTGGGACTAT |
| | CGCACCTCCGAGCTCGGCGACAACAAGTTCAATTTCACCACCAAATT |
| | CATCAATTTCTTTGAGCGCATCGGTCTCGCGTATGATCTCAAGACCG |
| | TTTCCGATGACGTTATCGCGCAACGCGCCAAACGCACCGGTGATGGT |
| | ACCCATCTCTGGGATTGCGCCGATAAGAATAATAACGATGTTGTTCA |
| | AACCAAAGCGCAAATCGATACCCTCTGCACCAAACATGAGTACCCCT |
| | ACGACGTGCCCGACTACGCCTAACATATGCCATGGTGTCAAAACCGG |
| | GGTTAGTGACATTGACTTGTTGACAAAAATCTGTATAGCTAGAAAAC |
| | TCTAAGCAACGCTTTTCTTTGTTTTATTTTTTATGTTTAAACTCCTT |
| | CAGAATTGTAGGATATCTTGTTTTGAAAAATCCAGGACTGAGTTTCG |
| | TTGCCCCATTTGCTTGTTCTCGTTTGAAATGTCGAACAATAGAAATG |
| | CTTGCAGAATGAGGTTCTCCTTTACAAAAAAACTCGATAGGGTTCAA |
| | TATGAAGCTGTCTCAATGCATAGATTTCCACGATTTTACCTTTGCAT |
| | AATCTATGGTGCGCGTCAGATGCCACCCTCGTCGCTGTACAACCAAT |
| | ACATTGTAGCTTCATTTTGACATTAGGTACCTTCTTCCCCGACCTCC |
| | TTCAGAATCTCAGAGTAAGCGATCGTCACCCCTTCTACCTGAAACTC |
| | TACCACTGCATACGTAGTAAAGGCCTCTAATTACCACGGTAGTACTA |
| | TTCTTGCACTGAGGAATTCTCTAGACGAATGTAGGCTATTCTTAATG |
| | GACCGGCCCTCAGCTCGATTATTTTTGCTTGACTTGACTTGACTTGA |
| | TTCATGAAGTTGATAGGAAAGAAACATAACCCATCCCATCCCACAAC |
| | CTGCGTGTACTCTGATCGGCAGGTGCACGCTGAGTTGAAGGTGGTTC |
| | AAGAATCGAAAACATCAGCCTAGAGCACGACGAGGTTTCAGAGAGCC |
| | AACTTTTTCTATCTATTAATCTCATCCTTTGCTTCTTCGCGGACAAC |
| | GACGGTGGATCAGCGCCGCCGCTGAGAAGACAGCAGAGGTAACTCTA |
| | GCAAGAGAAGCAGCAGTAGCTTCGTCTGGTCAAGAGACTCTGCTTAA |
| | GCACAGTAGCCTGCAAATAAAGACACTTGGGCAAAAGAAACATTGAC |
| | ATTGATTGAATTTCACGCAGAGGCAAATGGAAGCTT |

The nucleic acid sequence SEQ ID NO: 8 contains the nucleic acid sequence SEQ ID NO: 7 (shown in bold in Table 3) and allows the expression of the desaturase of amino acid sequence SEQ ID NO: 1 in Aurantiochytrium limacinum.

In another aspect, the invention relates to a vector comprising a nucleic acid as defined above or an expression cassette as defined above.

As used herein, a “vector” is a nucleic acid molecule used as a vehicle to transfer genetic material into a cell. The term “vector” encompasses plasmids, viruses, cosmids and artificial chromosomes. In general, engineered vectors comprise an origin of replication, a multicloning site and a selectable marker. The vector itself is generally a nucleotide sequence, commonly a DNA sequence, that comprises an insert (transgene) and a larger sequence that serves as the “backbone” of the vector. Modern vectors may encompass additional features besides the transgene insert and a backbone: promoter, genetic marker, antibiotic resistance, reporter gene, targeting sequence, protein purification tag. Vectors called expression vectors (expression constructs) specifically are for the expression of the transgene in the target cell, and generally have control sequences.

In another aspect, the invention relates to a protist comprising:
a nucleic acid as defined above,
an expression cassette as defined above, or
a vector as defined above.

A protist expressing the recombinant enzyme can be referred to as a “modified”, “transgenic” or “transformed” protist.

The invention furthermore relates to the use of a protist as defined above as feeds (for fisheries), as food supplements, cosmetic supplements or health supplements, for the production of polymers in green industry or for the production of biofuels.

The following figures and examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

### FIGURE LEGENDS

FIG. 1. Schematic representation of the plasmid pUbi-d11Tp encoding the Δ11 desaturase from T. pityocampa.

FIG. 2. Schematic representation of the plasmid pUbi-Zeo encoding the zeocin resistance.

FIG. 3. Schematic representation of the plasmid pUbi-d11Tp encoding the Δ11 desaturase from T. pseudonana.

FIG. 4. Dot plot of the sequence identity values calculated on a multialignment containing 484 delta11 desaturase sequences from the class Insecta. In x-axis: sequences, in y-axis: sequence identity value.

FIG. 5. Neighbor-Joining phylogenetic tree constructed with a subset of Δ11 sequences retrieved from NCBI. In the figure, the orders within the class Insecta of which the sequences belong are reported. For Lepidoptera, two groups have been identified: the butterflies and the moths. A black star identifies the Thaumetopoea pityocampa acyl-CoA Δ11 desaturase sequence.

FIG. 6. Fresh weight (A) and optical density at 600 nm (B) of the transgenic lines and control cultures after 5 days of culture run in parallel.

FIG. 7. Fatty Acid content in the transgenic lines and control cultures at days 2 and 5. The bold lines show the upper and lower range values for controls at day 5. Inset: picture of the chloroform extracted lipids in one of the transgenic lines (tube on the right) and a control (tube on the left). Note the different color intensities indicating a much higher oil content in the transgenic line.

FIG. 8. TAG content (A) and polar lipids (B) in control (empty vector) and four mutants overexpressing the Acyl-CoA Δ11 desaturase from the moth Thaumetopoea pityocampa.

FIG. 9. Fatty Acid composition (%) in transgenic lines and controls after 5 days of culture.

FIG. 10. DHA (22:6) and DPA (22:5) content in transgenic (dark grey) and control (light grey) cell lines.

FIG. 11. Dry weight of the transgenic lines and control cultures after 2 and 5 days of culture run in parallel.

FIG. 12. Fatty Acid content in the transgenic lines and control cultures at days 2 and 5.

FIG. 13. DPA (22:5) and DHA (22:6) content in transgenic (dark grey) and control (light grey) cell lines.

### EXAMPLES

### Materials and Methods

### Cultivation and Transformation of Aurantiochytrium

The thraustochytrid used in the examples is an Aurantiochytrium species (Aurantiochytrium limacinum). It was cultivated in R medium containing the ingredients listed in Table 5:

TABLE 5
Composition of the R medium.

| Component | Final Concentration (w/v) | |
|---|---|---|
| NaCl | 10.597 | g/l |
| Na₂SO₄ | 1.775 | g/l |
| NaHCO₃ | 87 | mg/l |
| KCl | 299.5 | mg/l |
| KBr | 43.15 | mg/l |
| H₃BO₃ | 11.5 | mg/l |
| NaF | 1.4 | mg/l |
| MgCl₂•6H₂O | 4.796 | g/l |
| CaCl₂•2H₂O | 0.672 | g/l |
| SrCl₂•6H₂O | 10.9 | mg/l |
| EDTA-iron | 1.50 | mg/l |
| Na₂EDTA•2H₂O | 1.545 | mg/l |
| ZnSO₄•7H₂O | 36.5 | μg/l |
| CoCl₂•6H₂O | 8 | μg/l |
| MnCl₂•4H₂O | 0.27 | mg/l |
| Na₂MoO₄•2H₂O | 0.74 | μg/l |
| Na₂Se0₃ | 0.085 | μg/l |
| NiCl₂•6H₂O | 0.745 | μg/l |
| CuSO₄•5H₂O | 4.9 | μg/l |
| Vitamin H | 0.499 | μg/l |
| Vitamin B12 | 0.501 | μg/l |
| Vitamin B1 | 78.54 | μg/l |
| NaNO₃ | 23.33 | mg/l |
| NaH₂PO₄ | 1.343 | mg/l |
| Glucose | 60 | g/l |
| Yeast extract | 20 | g/l |

50 ml cultures were grown in sterile 250 ml Pyrex flasks under agitation (100 rpm).

Solid medium has the same composition as Table 5 but contains 1% agar.

Cassette for the Expression of Acyl-CoA Δ11 Desaturase from Thaumetopoea pityocompa

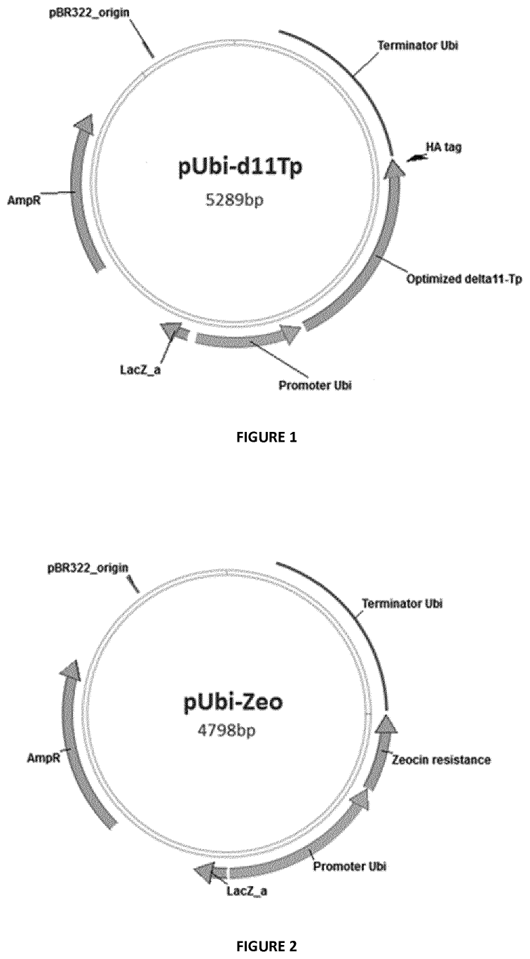

The polynucleotide coding for an acyl-CoA Δ11 desaturase from the moth Thaumetopoea pityocampa (SEQ ID NO: 1) was codon optimized using a homemade codon usage table (based on 30192 residues, see also Table 3) for heterologous over-expression in Aurantiochytrium under the control of the polyubiquitin endogenous gene promoter. The transcription terminator used in this construct was the endogenous polyubiquitin gene terminator. An HA tag sequence (YPYDVPDYA, SEQ ID NO: 9) was added between the last encoding amino acid and the stop codon of the acyl-CoA Δ11 desaturase sequence. Two restriction sites were added at the 5′ end (NotI, NheI) and the 3′ end (NdeI, NcoI) of the DNA sequence, producing the optimized delta11Tp-HA gene. The final delta11-Tp cassette (SEQ ID NO: 8), containing the Ubi promoter region from the pUbi-Zeo, followed by the optimized delta11Tp-HA gene, and the Ubi terminator region from the pUbi-Zeo, was synthesized and subcloned into a commercial pUC19 plasmid using PstI/HindIII restriction sites by the Invitrogen GeneArt Gene Synthesis Service to obtain the vector pUbi-d11Tp (FIG. 1). The plasmid was co-transformed with the zeocin resistance cassette under the same polyubiquitin promoter.

### Cassette for the Expression of the Zeocin Resistance

In order to clone the zeocin resistance gene under the polyubiquitin promoter/terminator into the expression vector, an ORF encoding a yeast UBI4 polyubiquitin homologous gene was identified in the genome of Aurantiochytrium. A 917 pb sequence upstream of the ORF was amplified with following primers PromUbi2SacI-F (TTGAGCTCAGAGCGCGAAAGAGAGTGCCGGAATTC, SEQ ID NO: 10)) and PromUbi2BamHI-R (GCGGATCCGAAATTGACCTTACTGCCTCTCCTGTG, SEQ ID NO: 11) to add the restriction sites SacI in 5′ and BamHI in 3′ of the sequence. A 935 pb sequence downstream of the ORF was amplified with the following primers TermUbi2SphI-F (GGGCATGCTGTCAAAACCGGGGTTAGTGACATTGA, SEQ ID NO: 12) and TermUbi2HindIII-R (GGAAGCTTCCATTTGCCTCTGCGTGAAATTCAATC, SEQ ID NO: 13) to add the restriction sites SphI in 5′ and HindIII in 3′ of the sequence. A 375 pb sequence encoding the zeocin gene from the commercial plasmid pTEF1 was amplified with following primers ZeoS1BamHI (GCGGATCCATGGCCAAGTTGACCAGTGCCGTTCC, SEQ ID NO: 14) and ZeoS1SalI (GCGTCGACTCAGTCCTGCTCCTCGGCCACGAAGT, SEQ ID NO: 15) to add the restriction sites BamHI in 5′ and SalI in 3′ of the sequence. All sequences were sequentially inserted into the multiple cloning site of the pUC19 plasmid to obtain the vector pUbi-Zeo (FIG. 2), using common cloning techniques (restriction enzyme digestion, ligation, E. coli termo-transformation, plasmid preparation). The sequence of the complete zeocin resistance cassette corresponds to SEQ ID NO: 16 (see Table 6).

TABLE 6
Cassette encoding the zeocin resistance.

| | |
|---|---|
| SEQ ID NO: 16 | GAGCTCAGAGCGCGAAAGAGAGTGCCGGATTCAAAGACGCCACAGCGGGAAAG |
| Cassette | AAAGAAAGACCTAGGAGGTACTAGCTGGTTGTAGCTAGCTAGCTAGCTAGCTA |
| encoding the | GCTTATGCTGCTAAGACGCCCTTCCTCCTCGAGGTCCTTTTGACTTGCCAGCG |
| zeocin resistance | CAGTCTCCTTTGTCTTCTTCGCTCATTTAATCAAGTCAAGTCTTCAGGTTTAA |
| | AATGAAAAATCCTGCTTCCAGGTTCAGTTCTAGCAAGTAGGTAGGTGGCAGTA |
| | GCGTTACGAGGAGGAGTCCCGAGAGGGAGTCGGAGAGTAGAAAACTGGAAGTC |
| | GGCGAAACAAAAGGCGCAGAGATTTGCCGGAATGGAGAGTTATCGTGAGACTC |
| | TCTGAGTAGACCCAAGTGTCCTGTGAGGCACTCGTGATAGGGAGGGGGCACGG |
| | GCTGAAGGGGGCTACAGTAAGGAGAGAGTGGCGTCAGTGGAGTTTCGCCGAGA |
| | ACTCTTCGAGAAAGAGGAAGAGAGGAACCGAGAGCGCCGTTGAAGAGGGGAAA |
| | AAGCAGACGGTTTAATTATAATTAATTAAGTAATTAATTACTTACTTATTGAT |
| | TGATTGATTTGAGAAGAGAAGCAAAGAGAGAGTTGAAGAAATAGTAACGAAGA |
| | ATAGGAGAAGAAAGGGGCAAGAAAAGAAAAAAGAAAGAGGAGAATATTGGTCG |
| | ATGAGCGAGAACGTGCAAATCCAAAACAGCAAAACTCAAACTCAAACTACAAG |
| | AAGCGTGGCGTTGCAGAGGCAACAGCTCGAAAGCAACACAGAACAGACAAACA |
| | CAGGAGAGGCAGTAAGGTCAATTTCGGATCCATGGCCAAGTTGACCAGTGCCG |
| | TTCCGGTGCTCACCGCGCGCGACGTCGCCGGAGCGGTCGAGTTCTGGACCGAC |
| | CGGCTCGGGTTCTCCCGGGACTTCGTGGAGGACGACTTCGCCGGTGTGGTCCG |
| | GGACGACGTGACCCTGTTCATCAGCGCGGTCCAGGACCAGGTGGTGCCGGACA |
| | ACACCCTGGCCTGGGTGTGGGTGCGCGGCCTGGACGAGCTGTACGCCGAGTGG |
| | TCGGAGGTCGTGTCCACGAACTTCCGGGACGCCTCCGGGCCGGCCATGACCGA |
| | GATCGGCGAGCAGCCGTGGGGGCAGGAGTTCGCCCTGCGCGACCCGGCCGGCA |
| | ACTGCGTGCACTTCGTGGCCGAGGAGCAGGACTGAGTCGACCTGCAGGCATGC |
| | TGTCAAAACCGGGGTTAGTGACATTGACTTGTTGACAAAAATCTGTATAGCTA |
| | GAAAACTCTAAGCAACGCTTTTCTTTGTTTTATTTTTTATGTTTTAACTCCTT |
| | CAGAATTGTAGGATATCTTGTTTTGAAAAATCCAGGACTGAGTTTCGTTGCCC |
| | CATTTGCTTGTTCTCGTTTGAAATGTCGAACAGTAGAAATGCTTGCAGAATGA |
| | GGTTCTCCTTTACAAAAAACTCGATAGGGTTCAATATGAAGCTGTCTCGATGC |
| | ATAGATTTCCACGATTTTACCTTTGCATAATCTATGGTGCGCGTCAGATGCCA |
| | CCCTCGTCGCTGTACAACCAATACATTGTAGCTTCATTTTGACATTAGGTACC |
| | TTCTTCCCCGACCTCCTTCAGAATCTCAGAGTAAGCGATCGATCGTCACCCCT |
| | TCTACCTGAAACTCTACCACTGCATACGTAGTAAAGGCCTCTAATTACCACGG |
| | TAGTACTATTCTTGCACTGAGGAATTCTCTAGACGAATGTAGGCTATTCTTAA |
| | TGGACCGGCCCTCAGCTCGATTATTTTTGCTTGACTTGACTTGACTTGATTAA |
| | TGAAGTTGATAGGAAAGAAACATAACCCATTCCATCCCACAACCTGCGTGTAC |
| | TCTGATCGGCAGGTGCACGCTGAGTTGAGGGTGATTTAAGGATCGAAAACATC |
| | AGCCTAGAGCACGACGAGGTTCCAGAGAGCCAACTTTTTCTATCTATTAATCT |
| | CATCCTTTGCTTCTTCGCGGACAACGACGGTGGATCAGCGCCGCCGCTGAGAA |
| | GACAGCAGAGGTAACTCTAGCAAGAGAAGCAGCAGTAGCTTCGTCTGGTCAAG |
| | AGACTCTGCTTAAGCACAGTAGCCTGCAAATAAAGACACTTGGGCAAAAGAAA |
| | CATTGACATTGATTGAATTTCACGCAGAGGCAAATGGAAGCTT |

Cassette for the Expression of an Acyl-CoA Δ11 Desaturase from Thalassiosira pseudonana

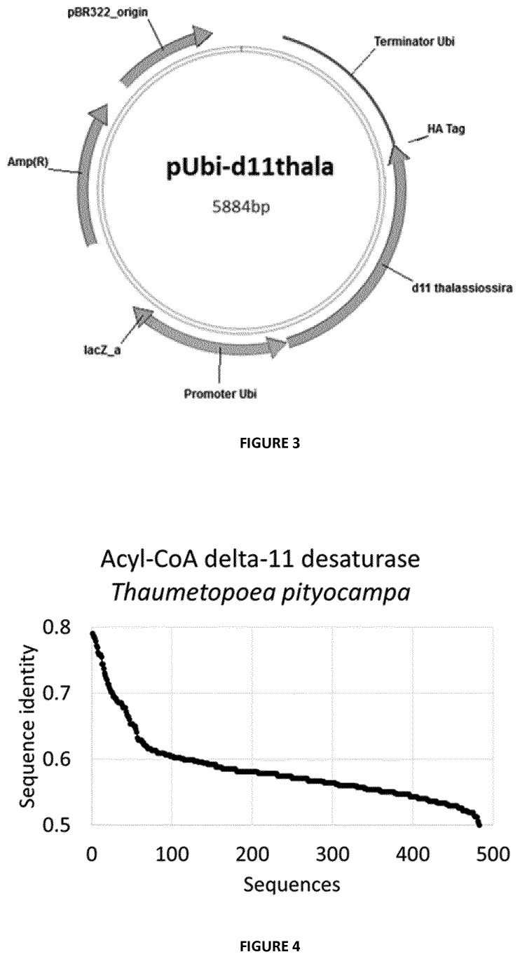

A sequence identified as a Δ11 desaturase (SEQ ID NO: 17, Thaps3|23391, see Table 7) was found in the genome of the marine diatom T. pseudonana. The nucleic acid sequence encoding this Δ11 desaturase was codon-optimized and synthesized as described above. Cloning into an expression vector was performed by GeneArt© gene synthesis service to obtain pUbi-d11thala (FIG. 3).

TABLE 7
Codon-optimized nucleic acid sequence encoding the Acyl-CoA Δ11
desaturase from Thalassiosira pseudonana.

| | |
|---|---|
| SEQ ID NO: 17 | CTGCAGGTAGGTAGGTGGCAGTAGCGTTACGAGGAGGAGTCCCGAGAGGGAGT |
| Cassette | CGGAGAGTAGAAAACTGGAAGTCGGCGAAACAAAAGGCGCAGAGATTTGCCGG |
| encoding a Acyl- | AATGGAGAGTTATCGTGAGACTCTCTGAGTAGACCCAAGTGTCCTGTGAGGCA |
| CoA Δ11 | CTCGTGATAGGGAGGGGGCACGGGCTGAAGGGGGCTACAGTAAGGAGAGAGTG |
| desaturase from | GCGTCAGTGGGGTTTTGCCGAGAACTCTTCGAGAAAGAGGAAGAGAGGAACCG |
| Thalassiosira | AGAGCGCCGTTGAAGAGGGGAAAAAGCAGACGGTTTAATTATAATTAATTAAG |
| pseudonana | TAATTAATTACTTACTTATTGATTGATTGATTTGAGAAGAGAAGCAAAGAGAG |
| | AGTTGAAGAAATAGTAACGAAGAATAGGAGAAGAAAGGGGCAAGAAAAGAAAA |
| | AGAAAGAGGAGAATATTAGTCGATGAGCGAGAACGTGCAAATCCAAAACAGCA |
| | AAACTCAAACTCAAACTCAAACTACAAGAAGCGTGGCGTTGCAGAGGCAACAG |
| | CTCGAAAGCAACACAGAACAAACAAACACAGGAGAGGCAGTAAGGTCAATTTC |
| | GCGGCCGCGCTAGCATGGCGCCCAACACGCGGGAGAACGAGACGATCTACGAC |
| | GAAGTGGAACACAAGCTGGAGAAGCTCGTGCCCCCCCAGGCGGGCCCCTGGAA |
| | CTACAAGATCGTGTACCTGAACTTGCTGACCTTCTCCTACTGGCTGATCGCCG |
| | GCGCCTACGGGTTGTACTTGTGCTTCACGTCCGCCAAGTGGGCCACGATCATC |
| | TTCGAATTCATCTTGTTCTTCTTCGCCGAGATGGGCATCACGGCCGGCGCCCA |
| | CCGGCTGTGGACGCACAAGTCCTACAAGGCCAAGTTGCCCTTGGAAATCTTCC |
| | TCATGGTGCTGAACTCCGTGGCGTTCCAGAACACGGCCACCGACTGGGTGCGG |
| | GACCACCGGCTGCACCACAAGTACAGCGACACGGACGCGGACCCCCACAACGC |
| | CGCGCGGGGGCTGTTCTTCTCCCACGTCGGGTGGCTGCTCGTCCGGAAGCACG |
| | ACGAAGTCAAGAAGCGCGGGAAGTTCACCGACATGTCCGACATCTACAACAAC |
| | CCCGTGTTGAAGTTCCAGAAGAAGTACGCCATCCCCTTCATCGGCGCCGTGTG |
| | CTTCATCTTGCCCACGGTGATCCCCATGTACTTCTGGGGCGAGTCCCTCAACA |
| | ACGCCTGGCACATCTGCATCCTGCGGTACGCGATGAACCTCAACGTCACGTTC |
| | TCCGTGAACTCCCTGGCGCACATCTGGGGCAACAAGCCCTACGACAAGGACAT |
| | CAAGCCCGCCCAGAACTTCGGCGTGACGTTGGCGACCTTCGGCGAAGGGTTCC |
| | ACAACTACCACCACGTGTTCCCCTGGGACTACCGGACGTCCGAACTCGGCGAC |
| | AACAAGTTCAACTTCACGACGAAGTTCATCAACTTCTTCGAACGGATCGGCTT |
| | GGCGTACGACCTGAAGACCGTGTCCGACGACGTGATCGCGCAGCGGGCCAAGC |
| | GGACCGGCGACGGCACGCACCTGTGGGACTGCGCCGACAAGAACAACAACGAC |
| | GTGGTGCAGACGAAGGCGCAGATCGACACCTTGTGCACGAAGCACGAATGAGG |
| | TTCTCCTTTACAAAAAAACTCGATAGGGTTCAATATGAAGCTGTCTCAATGCA |
| | TAGATTTCCACGATTTTACCTTTGCATAATCTATGGTGCGCGTCAGATGCCAC |
| | CCTCGTCGCTGTACAACCAATACATTGTAGCTTCATTTTGACATTAGGTACCT |
| | TCTTCCCCGACCTCCTTCAGAATCTCAGAGTAAGCGATCGTCACCCCTTCTAC |
| | CTGAAACTCTACCACTGCATACGTAGTAAAGGCCTCTAATTACCACGGTAGTA |
| | CTATTCTTGCACTGAGGAATTCTCTAGACGAATGTAGGCTATTCTTAATGGAC |
| | CGGCCCTCAGCTCGATTATTTTTGCTTGACTTGACTTGACTTGATTCATGAAG |
| | TTGATAGGAAAGAAACATAACCCATCCCATCCCACAACCTGCGTGTACTCTGA |
| | TCGGCAGGTGCACGCTGAGTTGAAGGTGGTTCAAGAATCGAAAACATCAGCCT |
| | AGAGCACGACGAGGTTTCAGAGAGCCAACTTTTTCTATCTATTAATCTCATCC |
| | TTTGCTTCTTCGCGGACAACGACGGTGGATCAGCGCCGCCGCTGAGAAGACAG |
| | CAGAGGTAACTCTAGCAAGAGAAGCAGCAGTAGCTTCGTCTGGTCAAGAGACT |
| | CTGCTTAAGCACAGTAGCCTGCAAATAAAGACACTTGGGCAAAAGAAACATTG |
| | ACATTGATTGAATTTCACGCAGAGGCAAATGGAAGCTT |

### Genetic Transformation

Genetic transformation was performed by biolistic method. 2×107 cells of Aurantiochytrium, from a 2 to 4-day old-culture, were plated onto solid medium with 200 μg/ml zeocin in 10 cm Petri dishes. Cells were left air-dry in a sterile hood. One to two μg of each plasmid for co-transformation were coated on 25 μl of 0.7 μm diameter tungsten microcarriers (hereon referred to as ‘beads’). 25 μL of CaCl2 2.5 M in absolute ethanol and 10 μL spermidine were added to the beads then 4 volumes of absolute ethanol to wash the beads. The beads were spun down for 6-7 sec at 8000 g, the supernatant discarded and 700 μl ice cold ethanol was added again. The supernatant was discarded and the pellet suspended in 25 μl ethanol. Coated beads were kept on ice until use. The particle bombardment was performed with a PDS-1000/He Particle Delivery System equipped with a rupture disk resistance 1550 psi. 10 μl of the bead mix was placed on the macrocarriers. Two shots per bead preparation were performed.

Genetic transformation of Aurantiochytrium can be achieved by other methods, such as electroporation.

### Lipid Extraction and Fatty Acid Analyses

Glycerolipids were extracted from freeze-dried cells. First, cells were harvested by centrifugation and snap-frozen in liquid nitrogen. Ten mg dry weight were suspended in 4 mL of boiling ethanol for 5 minutes. Lipids were extracted by addition of 2 mL methanol and 8 mL chloroform at room temperature (as described in Folch T et al., Journal of Biological Chemistry, 226:497-509, 1957). The mixture was saturated with argon and stirred for 1 hour at room temperature. After filtration through glass wool, cell remains were rinsed with 3 mL chloroform/methanol 2:1, v/v. Five mL of NaCl 1% were added to the filtrate to initiate biphase formation. The chloroform phase was dried under argon before solubilizing the lipid extract in pure chloroform (as described in Jouhet J et al., PLoS One, 12(8):e0182423, 2017).

Total fatty acids were analyzed as follows: in an aliquot fraction, a known quantity of 21:0 was added and the fatty acids present were converted to methyl esters (fatty acid methyl ester or FAME) by a 1-hour incubation in 3 mL 2.5% H2SO4 in pure methanol at 100° C. (as described in Jouhet et al., FEBS Letters, 544(1-3):63-8, 2003). The reaction was stopped by adding 3 mL 1:1 water:hexane. The hexane phase was analyzed by gas chromatography (gas chromatography coupled to mass spectrometry and flame ionization detection, GC-MS/FID) (Perkin Elmer, Clarus SQ 8 GC/MS series) on a BPX70 (SGE) column. FAMEs were identified by comparison of their retention times with those of standards (obtained from Sigma) and quantified using 21:0 for calibration. Extraction and quantification were performed at least 3 times.

### Quantification of Glycerolipids by High Performance Liquid Chromatography (HPLC) and Tandem Mass Spectrometry (MS/MS) Analyses

The various glycerolipids were routinely quantified using an external standard corresponding to a qualified control (QC) of lipids extracted from the same strain (as described in Jouhet J et al., PLoS One, 12(8):e0182423, 2017). This QC extract was a known lipid extract previously qualified and quantified by thin layer chromatography (TLC) and GC-MS/FID, as described above. For the routine analyses of the samples, lipids corresponding to 25 nmol of total fatty acids were dissolved in 100 μL of chloroform/methanol [2/1, (v/v)] containing 125 pmol each of DAG 18:0-22:6, PE 18:0-18:0 and SQDG 16:0-18:0 as internal standard (Avanti Polar Lipids Inc). All the internal standard solutions were first quantified by GC-FID. Lipids were then separated by HPLC and identified by ESI-MS/MS.

The HPLC separation method was adapted from Rainteau et al. (PLoS One, 7(7):e4198510, 2012). Lipid classes were separated using an Agilent 1200 HPLC system using a 150 mm×3 mm (length×internal diameter) 5 μm diol column (Macherey-Nagel), at 40° C. The mobile phases consisted of hexane/isopropanol/water/ammonium acetate 1M, pH5.3 [625/350/24/1, (v/v/v/v)] (A) and isopropanol/water/ammonium acetate 1M, pH5.3 [850/149/1, (v/v/v)] (B). The injection volume was 20 μL. After 5 min, the percentage of B was increased linearly from 0% to 100% in 30 min and stayed at 100% for 15 min. This elution sequence was followed by a return to 100% A in 5 min and an equilibration for 20 min with 100% A before the next injection, leading to a total runtime of 70 min. The flow rate of the mobile phase was 200 μL/min. The distinct glycerophospholipid classes were eluted successively as a function of the polar head group. Under these conditions, they were eluted in the following order: Triacylglycerols (TAG), Diacylglycerols (DAG), Phosphatidylethanolamines (PE), Phosphatidylglyecrols (PG), Phosphatidylinositols (PI), Phosphatidylserines (PS), Phosphatidylcholines (PC), Diphosphatidylglycerols (DPG) and Phosphatidic acids (PA).

Mass spectrometric analysis was done on a 6460 triple quadrupole mass spectrometer (Agilent) equipped with a Jet stream electrospray ion source under following settings: Drying gas heater: 260° C., Drying gas flow 13 L/min, Sheath gas heater: 300° C., Sheath gas flow: 11 L/min, Nebulizer pressure: 25 psi, Capillary voltage: ±5000 V, Nozzle voltage±1000. Nitrogen was used as collision gas. The quadrupoles Q1 and Q3 were operated at widest and unit resolution respectively. Mass spectra were processed by MassHunter Workstation software (Agilent). The QC sample is used as an external standard, and run with the list of the samples to be analyzed. First, lipid amounts in all samples were adjusted with three internal standards (see above) to correct possible variations linked to the injection and analytical run. Then, within the QC samples, molecules in a given class of glycerolipid were summed and compared to the amount of the same lipid class previously determined by TLC-GC. This is done in order to establish a correspondence between the area of the peaks and a number of pmoles. These corresponding factors were then applied to the samples of the list to be analyzed.

### Example I. Conservation of the Acyl-CoA Δ11 Desaturase Among Insects

A multialignment was carried out on 484 Δ11 sequences retrieved from the NCBI database using Thaumetopoea pityocampa acyl-CoA Δ11 desaturase as query (SEQ ID NO: 1). The sequences in fasta format were imported in BioEdit computer program and aligned using the ClustalW algorithm implemented in BioEdit. The alignment was trimmed in N-ter and C-ter taking into account the functional domains of the proteins. A sequence identity matrix was produced using the utility implemented in BioEdit software and the sequence identity values of Thaumetopoea pityocampa acyl-CoA Δ11 desaturase vs all the other 483 sequences in the alignment was plotted in FIG. 4. The 100% identity (value 1.00) of the comparison with the Thaumetopoea pityocampa acyl-CoA Δ11 desaturase sequence itself was not included in the dot plot. 23% (114) of the sequences presented an identity above 60%, 22% (107) an identity below 55%. All the amino acid sequences analyzed have a % identity equal or above 50% compared to SEQ ID NO: 1.

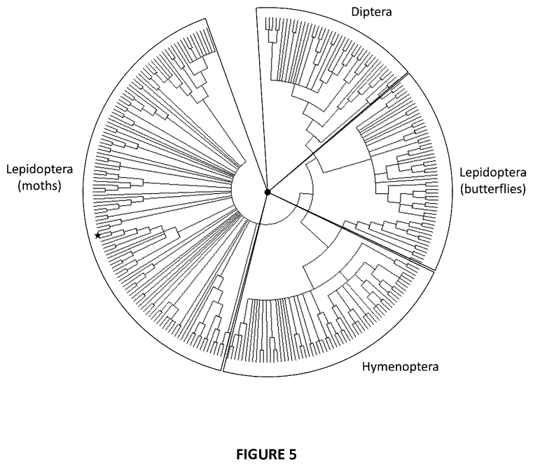

A subset of the alignment produced as described above, was used to construct a phylogenetic tree (FIG. 5). The evolutionary history was inferred using the Neighbor-Joining method. The bootstrap consensus tree inferred from 1000 replicates is taken to represent the evolutionary history of the taxa analyzed. Branches corresponding to partitions reproduced in less than 50% bootstrap replicates are collapsed. The evolutionary distances were computed using the Poisson correction method and are in the units of the number of amino acid substitutions per site. The rate variation among sites was modeled with a gamma distribution (shape parameter=1). The analysis involved 285 amino acid sequences. All ambiguous positions were removed for each sequence pair. There were a total of 300 positions in the final dataset. Evolutionary analyses were conducted in MEGA7.

### Example II. Production of Fatty Acids by Aurantiochytrium Clones Expressing the Acyl-CoA Δ11 Desaturase from Thaumetopoea pityocampa

Four transformant Aurantiochytrium clones, Thom7, Thom8, Thom10, Thom23′, obtained after transformation with the vector pUbi-d11Tp were PCR validated for the presence of the transgene in the genome and then used for the determination of their growth rate and lipid content. A wild type culture and a transformation control (pUbiZeo5) were added. The latter clone was transformed with the zeocin resistance cassette only and is meant to give the lipid baseline production for a biolistics-derived transformant. Growth was followed by measuring the fresh weight and the optical density at 600 nm of the cultures over a period of 5 days. Lipid measurements were performed on days 2 and 5. All the experiments were run in biological independent duplicates, except for pUbiZeo5 where two independent experiments were run, each in duplicate.

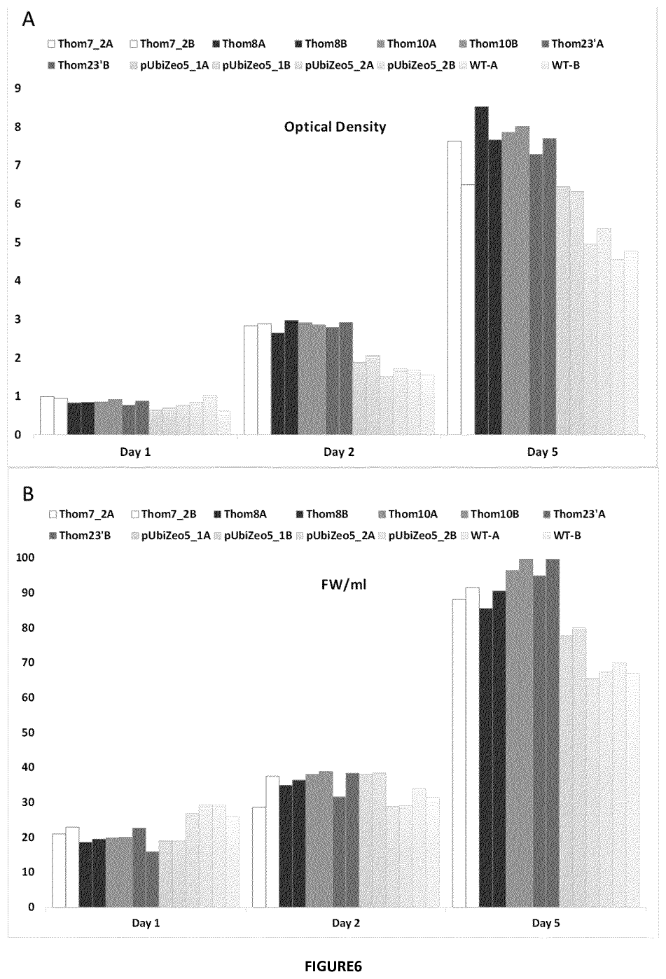

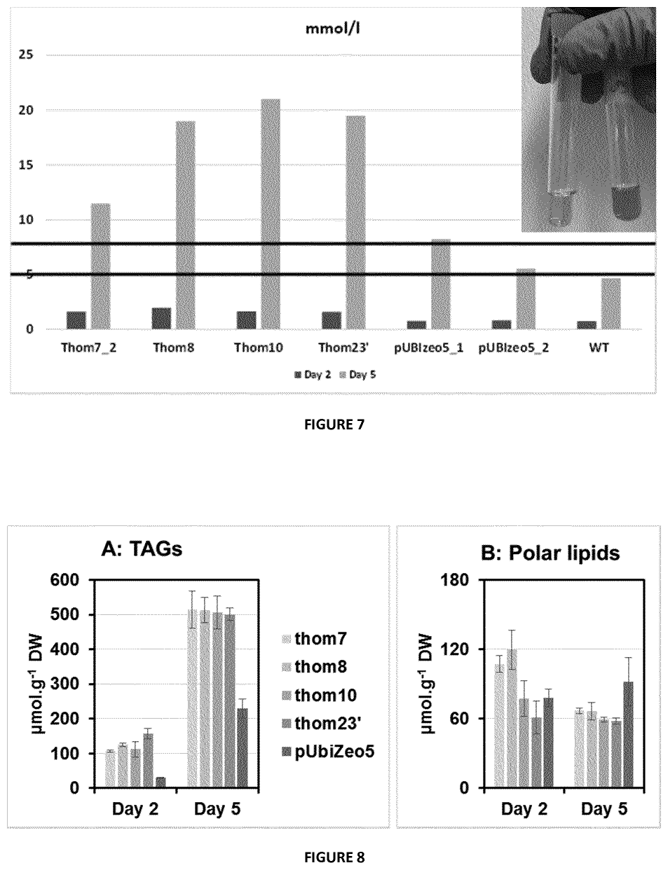

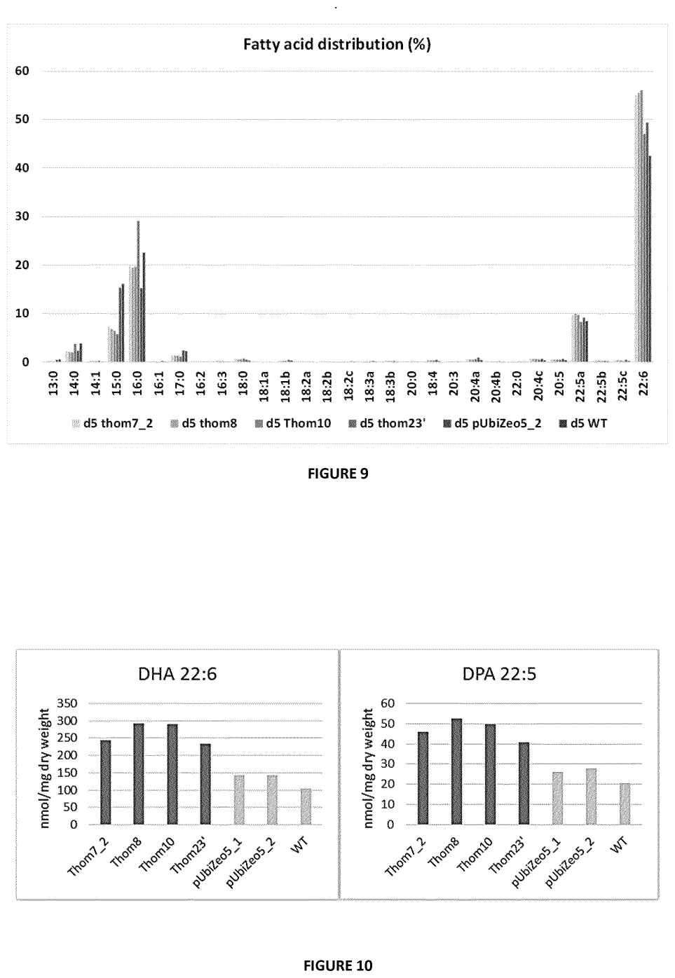

Fresh weight was comparable among transformants and controls during the first two days of the experiment, but at day 5 the four transformants had accumulated more biomass (higher fresh weight per milliliter of culture) and displayed a higher optical density (FIGS. 6A and 6B) than the controls. In addition, the four screened clones produced on average 2 times more total fatty acids per ml of culture (FIG. 7) than the controls on day 2 and 2-3 times more on day 5. The TAG content expressed as μmol per mg of fresh weight also increased by a factor of 2 (FIG. 8A), whereas the polar (membrane) lipid content was little affected (FIG. 8B), indicating that the increase of fatty acids was due to a higher level of lipid storage (TAGs). The FAME profiles of the transformants displayed a lower proportion of 15:0 compared to controls, and three transformants (Thom7, Thom8, Thom10) out of the four showed an augmented percentage of DHA. On average at day 5 the transgenic lines presented >54% DHA while the controls showed 45% (FIG. 9). In the transgenic lines Thom7, Thom8, Thom10, Thom23′, the PUFA content (DHA, 22:6 and DPA, 22:5) expressed per mg dry weight was on average twice as much as the controls at day 5 (FIG. 10) and, taking into account that they also produced more biomass (FIG. 6A), the yield of lipid production (μmoles/ml culture) was about three times higher.

This result shows that the overexpression of the Acyl-CoA Δ11 desaturase from Thaumetopoea pityocampa results in a higher rate of growth, improving the biomass production, together with an increase of total fatty acids and TAGs, without affecting the fatty acid composition.

### Example III. Production of Fatty Acids by Aurantiochytrium Clones Expressing the Acyl-CoA M1 Desaturase from Thalassiosira pseudonana (Comparative Example)

In WO 2005/080578, desaturases from the diatom Thalassiosira pseudonana were identified (TpDESN) and functionally characterized in T. pseudonana and in yeast. By supplementing the culture media with different fatty acids, it was possible to identify such a Δ11 desaturase as not being a front-end desaturase albeit its primary sequence shows high similarity with this protein family. TpDESN acts primarily on 16:0. The expression of this protein in the yeast led to the production of specific fatty acids upon culture medium supplementation with different fatty acid substrates.

A sequence identified as a Δ11 desaturase (SEQ ID NO: 17, Thaps3|23391) was found in the genome of the marine diatom T. pseudonana. Aurantiochytrium was transformed to express this Δ11 desaturase. This Δ11 desaturase showed 10.6% homology with the Thaumetopoea pityocampa acyl-CoA Δ11 desaturase of Example 2.

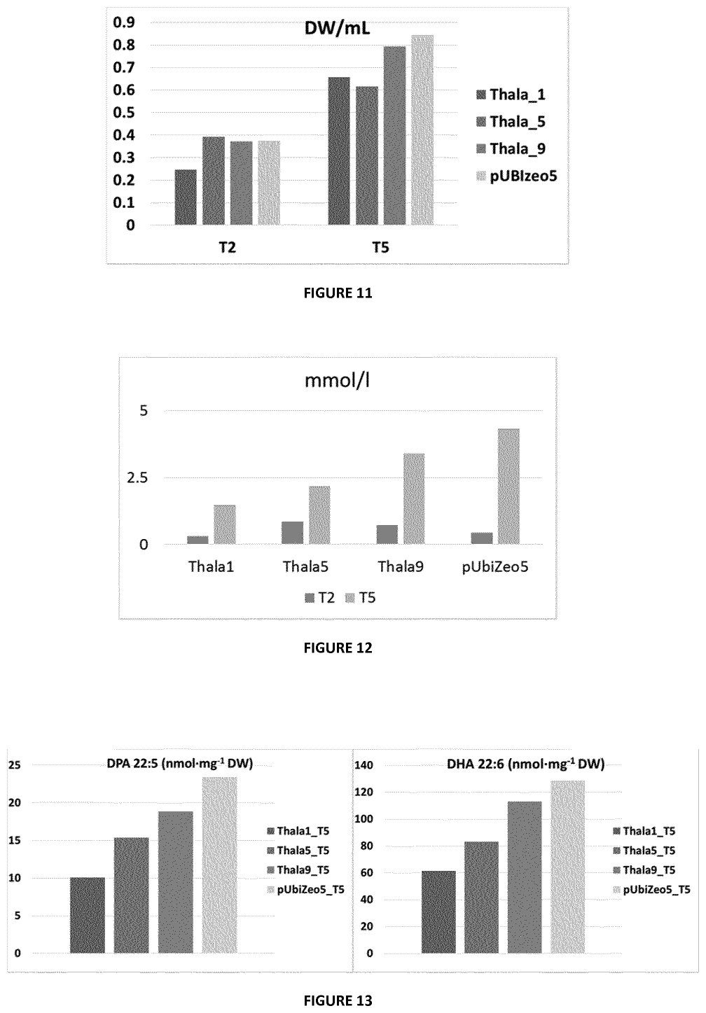

Three transformant Aurantiochytrium clones were analyzed, Thala1, Thala5, and Thala9. Growth and biomass accumulation was slightly affected in all the transformants compared to the pUbiZeo5 negative control (FIG. 11).

The total fatty acid production was affected in transformant clones (FIG. 12) as well as the DHA and DPA content (FIG. 13), showing reduced fatty acid and PUFA contents.

1. (canceled)

2. A method for producing triacylglycerides and/or fatty acids, expressing a recombinant fatty acid Δ11 desaturase in a protist, wherein said recombinant fatty acid Δ11 desaturase comprises or consists of a sequence having at least 50% identity with the sequence SEQ ID NO: 1.

3. The method of claim 2, wherein said recombinant fatty acid Δ11 desaturase comprises or consists of the sequence SEQ ID NO: 1, SEQ ID NO: 2 or SEQ ID NO: 3.

4. The method of claim 2, wherein said protist is a microalgae.

5. The method of claim 2, wherein said protist is a Thraustochytrid.

6. The method of claim 2, wherein said fatty acids are polyunsaturated fatty acids.

7. The method of claim 2, wherein said fatty acids are eicosapentaenoic acid (EPA, 20:5), docosapentaenoic acid (DPA, 22:5) or docosahexaenoic acid (DHA, 22:6).

8. A nucleic acid encoding a fatty acid Δ11 desaturase comprising or consisting of a sequence having at least 50% identity with the sequence SEQ ID NO: 1, said nucleic acid being codon-optimized for the expression of said fatty acid Δ11 desaturase in a protist.

9. The nucleic acid according to claim 8 which comprises or consists of the sequence SEQ ID NO: 7.

10. An expression cassette comprising a nucleic acid encoding a fatty acid Δ11 desaturase as recited in claim 8, said nucleic acid encoding a fatty acid Δ11 desaturase being under the control of a promoter which is functional in a protist.

11. A vector comprising a nucleic acid as defined in claim 8 or comprising an expression cassette comprising said nucleic acid, wherein said nucleic acid in the expression cassette is under the control of a promoter which is functional in a protist.

12. A protist comprising:
a nucleic acid as defined in claim 8,
an expression cassette comprising said nucleic acid, wherein said nucleic acid in the expression cassette is under the control of a promoter which is functional in a protist, or
a vector comprising said nucleic acid or said expression cassette.

13. The method of claim 5, wherein said Thraustochytrid is from a genus selected from the group consisting of Aurantiochytrium, Japonochytrium, Sicyoidochytrium, Ulkenia, Parietichytrium, Botryochytrium, Schizochytrium, Monorhizochytrium and Thraustochytrium.

14. The method of claim 5, wherein said Thraustochytrid is selected from the species Aurantiochytrium limacinum and Aurantiochytrium mangrovei.

15. The method of claim 6, wherein said fatty acids are long-chain polyunsaturated fatty acids or very long-chain polyunsaturated fatty acids.


SEQUENCE LISTING (17 sequences)

SEQ ID NO: 1 - protein, 347 aa, Thaumetopoea pityocampa
MAPNTRENETIYDEVEHKLEKLVPPQAGPWNYKIVYLNLLTFSYWLIAGAYGLYLCFTSAKWATIIFEFILFFFAEMGITAGAHRLWTHKSYKAKLPLEIFLMVLNSVAFQNTATDWVRDHRLHHKYSDTDADPHNAARGLFFSHVGWLLVRKHDEVKKRGKFTDMSDIYNNPVLKFQKKYAIPFIGAVCFILPTVIPMYFWGESLNNAWHICILRYAMNLNVTFSVNSLAHIWGNKPYDKDIKPAQNFGVTLATFGEGFHNYHHVFPWDYRTSELGDNKFNFTTKFINFFERIGLAYDLKTVSDDVIAQRAKRTGDGTHLWDCADKNNNDVVQTKAQIDTLCTKHE

SEQ ID NO: 2 - protein, 338 aa, Helicoverpa zea
MAQSYQSTTVLSEEKELTLQHLVPQASPRKYQIVYPNLITFGYWHIAGLYGLYLCFTSAKWATILFSYILFVLAEIGITAGAHRLWAHKTYKAKLPLEILLMVFNSIAFQNSAIDWVRDHRLHHKYSDTDADPHNASRGFFYSHVGWLLVRKHPEVKKRGKELNMSDIYNNPVLRFQKKYAIPFIGAVCFALPTMIPVYFWGETWSNAWHITMLRYIMNLNVTFLVNSAAHIWGNKPYDAKILPAQNVAVSVATGGEGFHNYHHVFPWDYRAAELGNNSLNLTTKFIDLFAAIGWAYDLKTVSEDMIKQRIKRTGDGTDLWGHEQNCDEVWDVKDKSS

SEQ ID NO: 3 - protein, 338 aa, Spodoptera littoralis
MAQCVQTTTILEQKEEKTVTLLVPQAGKRKFEIVYFNIITFAYWHIAGLYGLYLCFTSTKWATVLFSFFLFVVAEVGVTAGSHRLWSHKTYKAKLPLQILLMVMNSLAFQNTVIDWVRDHRLHHKYSDTDADPHNASRGFFYSHVGWLLVRKHPDVKKRGKEIDISDIYNNPVLRFQKKYAIPFIGAVCFVLPTLIPVYGWGETWTNAWHVAMLRYIMNLNVTFLVNSAAHIYGKRPYDKKILPSQNIAVSIATFGEGFHNYHHVFPWDYRAAELGNNSLNFPTKFIDFFAWIGWAYDLKTVSKEMIKQRSKRTGDGTNLWGLEDVDTPEDLKNTKGE

SEQ ID NO: 4 - protein, 6 aa, Artificial sequence
  Consensus sequence of His box
  MISC_FEATURE (5)..(5): X = T or A or S
HRLWXH

SEQ ID NO: 5 - protein, 7 aa, Artificial sequence
  Consensus sequence of His box
  MISC_FEATURE (1)..(1): X = D or E
  MISC_FEATURE (4)..(4): X = L or M or F or S
  MISC_FEATURE (7)..(7): X = K or R
XHRXHHX

SEQ ID NO: 6 - protein, 7 aa, Artificial sequence
  Consensus sequence of His box
  MISC_FEATURE (1)..(1): X = F or S
  MISC_FEATURE (7)..(7): X = V or T
XHNYHHX

SEQ ID NO: 7 - DNA, 1295 bp, Artificial sequence
  Optimized sequence for the expression of a delta11 desaturase in Aurantiochytrium
atggcgcccaacacccgcgagaacgagaccatctacgatgaggttgagcataagctcgagaaactcgtgcctcctcaagcgggcccctggaactacaaaatcgtttatctcaaccttctcaccttctcttactggcttatcgccggcgcctacggtctctatctctgttttacctccgcaaaatgggccaccatcatcttcgagttcatcctcttctttttcgccgagatgggcatcaccgcaggtgctcaccgcctctggacccataaatcttacaaagccaagctccctctcgagatcttcctcatggtgctcaattctgttgcgttccaaaacacggccacggactgggtgcgcgatcatcgcctccaccataagtactccgacaccgacgcggatcctcacaacgctgcgcgcggtctcttcttctcccatgtcggttggctcctcgtccgcaagcacgacgaggtcaagaagcgcggtaagtttaccgatatgtccgatatctacaacaatcccgtgctcaagttccagaagaaatatgccatccccttcatcggtgccgtttgctttatcttacctaccgtgatccccatgtacttttggggtgagtccctcaacaacgcctggcacatctgtatcctccgctatgcgatgaacctcaacgtcaccttctccgtgaactccctcgcgcatatctggggtaataagccctacgacaaggatatcaaacccgctcagaacttcggtgttaccctcgcgaccttcggtgagggttttcacaactatcaccacgtgttcccctgggactatcgcacctccgagctcggcgacaacaagttcaatttcaccaccaaattcatcaatttctttgagcgcatcggtctcgcgtatgatctcaagaccgtttccgatgacgttatcgcgcaacgcgccaaacgcaccggtgatggtacccatctctgggattgcgccgataagaataataacgatgttgttcaaaccaaagcgcaaatcgataccctctgcaccaaacatgagtacccctacgacgtgcccgactacgcctaacatatgccatggtgtcaaaaccggggttagtgacattgacttgttgacaaaaatctgtatagctagaaaactctaagcaacgcttttctttgttttattttttatgtttaaactccttcagaattgtaggatatcttgttttgaaaaatccaggactgagtttcgttgccccatttgcttgttctcgtttgaaatgtcgaacaatagaaatgcttgcagaatga

SEQ ID NO: 8 - DNA, 2621 bp, Artificial sequence
  Recombinant cassette for the expression of the delta11 desaturase
ctgcaggtaggtaggtggcagtagcgttacgaggaggagtcccgagagggagtcggagagtagaaaactggaagtcggcgaaacaaaaggcgcagagatttgccggaatggagagttatcgtgagactctctgagtagacccaagtgtcctgtgaggcactcgtgatagggagggggcacgggctgaagggggctacagtaaggagagagtggcgtcagtggggttttgccgagaactcttcgagaaagaggaagagaggaaccgagagcgccgttgaagaggggaaaaagcagacggtttaattataattaattaagtaattaattacttacttattgattgattgatttgagaagagaagcaaagagagagttgaagaaatagtaacgaagaataggagaagaaaggggcaagaaaagaaaaagaaagaggagaatattagtcgatgagcgagaacgtgcaaatccaaaacagcaaaactcaaactcaaactcaaactacaagaagcgtggcgttgcagaggcaacagctcgaaagcaacacagaacaaacaaacacaggagaggcagtaaggtcaatttcgcggccgcgctagcatggcgcccaacacccgcgagaacgagaccatctacgatgaggttgagcataagctcgagaaactcgtgcctcctcaagcgggcccctggaactacaaaatcgtttatctcaaccttctcaccttctcttactggcttatcgccggcgcctacggtctctatctctgttttacctccgcaaaatgggccaccatcatcttcgagttcatcctcttctttttcgccgagatgggcatcaccgcaggtgctcaccgcctctggacccataaatcttacaaagccaagctccctctcgagatcttcctcatggtgctcaattctgttgcgttccaaaacacggccacggactgggtgcgcgatcatcgcctccaccataagtactccgacaccgacgcggatcctcacaacgctgcgcgcggtctcttcttctcccatgtcggttggctcctcgtccgcaagcacgacgaggtcaagaagcgcggtaagtttaccgatatgtccgatatctacaacaatcccgtgctcaagttccagaagaaatatgccatccccttcatcggtgccgtttgctttatcttacctaccgtgatccccatgtacttttggggtgagtccctcaacaacgcctggcacatctgtatcctccgctatgcgatgaacctcaacgtcaccttctccgtgaactccctcgcgcatatctggggtaataagccctacgacaaggatatcaaacccgctcagaacttcggtgttaccctcgcgaccttcggtgagggttttcacaactatcaccacgtgttcccctgggactatcgcacctccgagctcggcgacaacaagttcaatttcaccaccaaattcatcaatttctttgagcgcatcggtctcgcgtatgatctcaagaccgtttccgatgacgttatcgcgcaacgcgccaaacgcaccggtgatggtacccatctctgggattgcgccgataagaataataacgatgttgttcaaaccaaagcgcaaatcgataccctctgcaccaaacatgagtacccctacgacgtgcccgactacgcctaacatatgccatggtgtcaaaaccggggttagtgacattgacttgttgacaaaaatctgtatagctagaaaactctaagcaacgcttttctttgttttattttttatgtttaaactccttcagaattgtaggatatcttgttttgaaaaatccaggactgagtttcgttgccccatttgcttgttctcgtttgaaatgtcgaacaatagaaatgcttgcagaatgaggttctcctttacaaaaaaactcgatagggttcaatatgaagctgtctcaatgcatagatttccacgattttacctttgcataatctatggtgcgcgtcagatgccaccctcgtcgctgtacaaccaatacattgtagcttcattttgacattaggtaccttcttccccgacctccttcagaatctcagagtaagcgatcgtcaccccttctacctgaaactctaccactgcatacgtagtaaaggcctctaattaccacggtagtactattcttgcactgaggaattctctagacgaatgtaggctattcttaatggaccggccctcagctcgattatttttgcttgacttgacttgacttgattcatgaagttgataggaaagaaacataacccatcccatcccacaacctgcgtgtactctgatcggcaggtgcacgctgagttgaaggtggttcaagaatcgaaaacatcagcctagagcacgacgaggtttcagagagccaactttttctatctattaatctcatcctttgcttcttcgcggacaacgacggtggatcagcgccgccgctgagaagacagcagaggtaactctagcaagagaagcagcagtagcttcgtctggtcaagagactctgcttaagcacagtagcctgcaaataaagacacttgggcaaaagaaacattgacattgattgaatttcacgcagaggcaaatggaagctt

SEQ ID NO: 9 - protein, 9 aa, Artificial sequence
  HA tag sequence
YPYDVPDYA

SEQ ID NO: 10 - DNA, 35 bp, Artificial sequence
  Primer
ttgagctcagagcgcgaaagagagtgccggaattc

SEQ ID NO: 11 - DNA, 35 bp, Artificial sequence
  Primer
gcggatccgaaattgaccttactgcctctcctgtg

SEQ ID NO: 12 - DNA, 35 bp, Artificial sequence
  Primer
gggcatgctgtcaaaaccggggttagtgacattga

SEQ ID NO: 13 - DNA, 35 bp, Artificial sequence
  Primer
ggaagcttccatttgcctctgcgtgaaattcaatc

SEQ ID NO: 14 - DNA, 34 bp, Artificial sequence
  Primer
gcggatccatggccaagttgaccagtgccgttcc

SEQ ID NO: 15 - DNA, 34 bp, Artificial sequence
  Primer
gcgtcgactcagtcctgctcctcggccacgaagt

SEQ ID NO: 16 - DNA, 2163 bp, Artificial sequence
  Optimized sequence for the expression of the zeocin resistance in Aurantiochytrium
gagctcagagcgcgaaagagagtgccggattcaaagacgccacagcgggaaagaaagaaagacctaggaggtactagctggttgtagctagctagctagctagctagcttatgctgctaagacgcccttcctcctcgaggtccttttgacttgccagcgcagtctcctttgtcttcttcgctcatttaatcaagtcaagtcttcaggtttaaaatgaaaaatcctgcttccaggttcagttctagcaagtaggtaggtggcagtagcgttacgaggaggagtcccgagagggagtcggagagtagaaaactggaagtcggcgaaacaaaaggcgcagagatttgccggaatggagagttatcgtgagactctctgagtagacccaagtgtcctgtgaggcactcgtgatagggagggggcacgggctgaagggggctacagtaaggagagagtggcgtcagtggagtttcgccgagaactcttcgagaaagaggaagagaggaaccgagagcgccgttgaagaggggaaaaagcagacggtttaattataattaattaagtaattaattacttacttattgattgattgatttgagaagagaagcaaagagagagttgaagaaatagtaacgaagaataggagaagaaaggggcaagaaaagaaaaaagaaagaggagaatattggtcgatgagcgagaacgtgcaaatccaaaacagcaaaactcaaactcaaactacaagaagcgtggcgttgcagaggcaacagctcgaaagcaacacagaacagacaaacacaggagaggcagtaaggtcaatttcggatccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcaggagttcgccctgcgcgacccggccggcaactgcgtgcacttcgtggccgaggagcaggactgagtcgacctgcaggcatgctgtcaaaaccggggttagtgacattgacttgttgacaaaaatctgtatagctagaaaactctaagcaacgcttttctttgttttattttttatgttttaactccttcagaattgtaggatatcttgttttgaaaaatccaggactgagtttcgttgccccatttgcttgttctcgtttgaaatgtcgaacagtagaaatgcttgcagaatgaggttctcctttacaaaaaactcgatagggttcaatatgaagctgtctcgatgcatagatttccacgattttacctttgcataatctatggtgcgcgtcagatgccaccctcgtcgctgtacaaccaatacattgtagcttcattttgacattaggtaccttcttccccgacctccttcagaatctcagagtaagcgatcgatcgtcaccccttctacctgaaactctaccactgcatacgtagtaaaggcctctaattaccacggtagtactattcttgcactgaggaattctctagacgaatgtaggctattcttaatggaccggccctcagctcgattatttttgcttgacttgacttgacttgattaatgaagttgataggaaagaaacataacccattccatcccacaacctgcgtgtactctgatcggcaggtgcacgctgagttgagggtgatttaaggatcgaaaacatcagcctagagcacgacgaggttccagagagccaactttttctatctattaatctcatcctttgcttcttcgcggacaacgacggtggatcagcgccgccgctgagaagacagcagaggtaactctagcaagagaagcagcagtagcttcgtctggtcaagagactctgcttaagcacagtagcctgcaaataaagacacttgggcaaaagaaacattgacattgattgaatttcacgcagaggcaaatggaagctt

SEQ ID NO: 17 - DNA, 2370 bp, Artificial sequence
  Optimized sequence for the expression of a delta11 desaturase in Aurantiochytrium
ctgcaggtaggtaggtggcagtagcgttacgaggaggagtcccgagagggagtcggagagtagaaaactggaagtcggcgaaacaaaaggcgcagagatttgccggaatggagagttatcgtgagactctctgagtagacccaagtgtcctgtgaggcactcgtgatagggagggggcacgggctgaagggggctacagtaaggagagagtggcgtcagtggggttttgccgagaactcttcgagaaagaggaagagaggaaccgagagcgccgttgaagaggggaaaaagcagacggtttaattataattaattaagtaattaattacttacttattgattgattgatttgagaagagaagcaaagagagagttgaagaaatagtaacgaagaataggagaagaaaggggcaagaaaagaaaaagaaagaggagaatattagtcgatgagcgagaacgtgcaaatccaaaacagcaaaactcaaactcaaactcaaactacaagaagcgtggcgttgcagaggcaacagctcgaaagcaacacagaacaaacaaacacaggagaggcagtaaggtcaatttcgcggccgcgctagcatggcgcccaacacgcgggagaacgagacgatctacgacgaagtggaacacaagctggagaagctcgtgcccccccaggcgggcccctggaactacaagatcgtgtacctgaacttgctgaccttctcctactggctgatcgccggcgcctacgggttgtacttgtgcttcacgtccgccaagtgggccacgatcatcttcgaattcatcttgttcttcttcgccgagatgggcatcacggccggcgcccaccggctgtggacgcacaagtcctacaaggccaagttgcccttggaaatcttcctcatggtgctgaactccgtggcgttccagaacacggccaccgactgggtgcgggaccaccggctgcaccacaagtacagcgacacggacgcggacccccacaacgccgcgcgggggctgttcttctcccacgtcgggtggctgctcgtccggaagcacgacgaagtcaagaagcgcgggaagttcaccgacatgtccgacatctacaacaaccccgtgttgaagttccagaagaagtacgccatccccttcatcggcgccgtgtgcttcatcttgcccacggtgatccccatgtacttctggggcgagtccctcaacaacgcctggcacatctgcatcctgcggtacgcgatgaacctcaacgtcacgttctccgtgaactccctggcgcacatctggggcaacaagccctacgacaaggacatcaagcccgcccagaacttcggcgtgacgttggcgaccttcggcgaagggttccacaactaccaccacgtgttcccctgggactaccggacgtccgaactcggcgacaacaagttcaacttcacgacgaagttcatcaacttcttcgaacggatcggcttggcgtacgacctgaagaccgtgtccgacgacgtgatcgcgcagcgggccaagcggaccggcgacggcacgcacctgtgggactgcgccgacaagaacaacaacgacgtggtgcagacgaaggcgcagatcgacaccttgtgcacgaagcacgaatgaggttctcctttacaaaaaaactcgatagggttcaatatgaagctgtctcaatgcatagatttccacgattttacctttgcataatctatggtgcgcgtcagatgccaccctcgtcgctgtacaaccaatacattgtagcttcattttgacattaggtaccttcttccccgacctccttcagaatctcagagtaagcgatcgtcaccccttctacctgaaactctaccactgcatacgtagtaaaggcctctaattaccacggtagtactattcttgcactgaggaattctctagacgaatgtaggctattcttaatggaccggccctcagctcgattatttttgcttgacttgacttgacttgattcatgaagttgataggaaagaaacataacccatcccatcccacaacctgcgtgtactctgatcggcaggtgcacgctgagttgaaggtggttcaagaatcgaaaacatcagcctagagcacgacgaggtttcagagagccaactttttctatctattaatctcatcctttgcttcttcgcggacaacgacggtggatcagcgccgccgctgagaagacagcagaggtaactctagcaagagaagcagcagtagcttcgtctggtcaagagactctgcttaagcacagtagcctgcaaataaagacacttgggcaaaagaaacattgacattgattgaatttcacgcagaggcaaatggaagctt